(19) United States
(12) Patent Application Publication
(10) Pub. No.: US 2024/0060141 A1
(43) Pub. Date: Feb. 22, 2024

(54) DETECTION OF LUNG CANCER USING CELL-FREE DNA FRAGMENTATION

(71) Applicant: The Johns Hopkins University, Baltimore, MD (US)

(72) Inventors: Victor Velculescu, Glenwood, MD (US); Robert B. Scharpf, Baltimore, MD (US); Dimitrios Mathios, Baltimore, MD (US); Jillian A. Phallen, Baltimore, MD (US); Daniel Bruhm, Baltimore, MD (US); Stephen Cristiano, Blatimore, MD (US)

(21) Appl. No.: 18/268,893

(22) PCT Filed: Dec. 21, 2021
(86) PCT No.: PCT/US21/64613

§ 371 Date: Jun. 21, 2023

Related U.S. Application Data
(60) Provisional application No. 63/197,301, filed on Jun. 4, 2021.
(60) Provisional application No. 63/128,776, filed on Dec. 21, 2020.

(51) Int. Cl. C12Q 1/6886; C12Q 1/6806; C12Q 1/6869; G01N 33/68; G16B 30/10; G16H 50/20; G16H 50/70

(52) U.S. Cl. CPC C12Q 1/6886; C12Q 1/6806; C12Q 1/6869; G01N 33/6893; G16B 30/10; G16H 50/20; G16H 50/70; C12Q 2600/112; C12Q 2600/156

(57) ABSTRACT

Cell free DNA (cfDNA) fragmentation for lung cancer detection is combined with current imaging-based screening methods and biomarkers.

The present application claims the benefit of priority of 1) U.S. provisional application No. 63/128,776, filed Dec. 21, 2020 and 2) U.S. provisional application No. 63/197,301, filed Jun. 4, 2021, each of which applications is incorporated by reference herein in their entirety.

### STATEMENT REGARDING FEDERALLY SPONSORED RESEARCH

This invention was made with government support under grants CA121113 and CA233259 awarded by the National Institutes of Health. The government has certain rights in the invention.

### FIELD

The present disclosure relates in general to methods for detecting and diagnosing cancer, in particular lung cancer, at early stages of the disease.

### BACKGROUND

Lung cancer is the most lethal cancer in the world¹. The 5-year survival rate is less than 20%² largely due to the late stage at diagnosis where treatments are less effective than at earlier stages, and the incidence of lung cancer continues to increase worldwide³. Although large randomized trials have demonstrated that lung cancer screening using chest low dose computed tomography (LDCT) decreases mortality in high risk individuals^{4,5}, LDCT remains underutilized, with less than 6% of at-risk individuals screened, due to concerns of potential harm from false positive imaging results, radiation exposure, and morbidity from invasive diagnostic procedures⁶⁻⁸.

### SUMMARY

We now provide new non-invasive methods for detecting and diagnosing cancer, in particular lung cancer, at early stages of the disease. Accordingly, in certain embodiments, a method of diagnosing cancer in a subject, comprises extracting cell free (cfDNA) from the subject's biological sample; generating genomic libraries from the extracted cfDNA and whole genome sequencing of cfDNA fragments; mapping of the cfDNA fragments to a genomic origin and evaluating fragment length and obtaining genome-wide fragmentation profiles for each sample; identifying protein biomarkers of the subject; comparing the subject's cfDNA fragmentation profile and protein biomarkers with normal reference non-cancer subjects. In certain embodiments, the cancer is lung cancer.

In certain embodiments, the method further comprises subjecting the subject to low dose helical computed tomography (LDCT). In certain embodiments, the method further comprises comparing clinical data between the subject diagnosed as having lung cancer and non-cancer subjects. In certain embodiments, the cfDNA fragment mean length and profiles are similar among non-cancer individuals. In certain embodiments, the cfDNA fragment profiles of cancer subjects vary. In certain embodiments, the serum levels of or one or more tumor antigens, cytokines or proteins are measured.

In certain embodiments, the one or more tumor antigens comprise: carcinoembryonic antigen (CEA), CA19-9, CA 125, tissue polypeptideantigen (TSA), CYFRA-21-1, neuron-specific enolase, progastrin-releasing peptide (ProGRP), plasma kalikrein B1 (KLKB1), serum amyloid A, haptoglobin-alpha-2, ADAM-17, osteoprotegerin, pentraxin 3, follistatin, tumor necrosis factor receptor superfamily member 1A or combinations thereof.

In certain embodiments, the one or more proteins comprise C-reactive protein (CRP), Chitinase-3-like protein 1 (YKL-40/CHI3L1) or fragments thereof.

In certain embodiments a DELFI (DNA evaluation of fragments for early interception) score is generated, wherein the principle component analysis is incorporated into a machine learning predictive model to generate a score for each subject as an average over cross-validation repeats (DELFI score(s)). As an example, due to the high dimensionality of the fragmentation features relative to the number of available samples for training, a principal component analysis was performed within each training set to reduce the dimensionality of the feature space, retaining the minimum number of principal components needed to explain 90% of the variance of the fragmentation profiles between samples. In addition to the principal component features, all 39 z-scores were evaluated in a logistic regression model with a LASSO penalty. The optimized LASSO penalty in our analysis was obtained by resampling using the caret R package. The DELFI score derived for each sample corresponds to the mean score across the 10 cross validation repeats. References herein to “DELFI score” are values determined by this above specified procedure.

In certain embodiments, the DELFI scores for non-cancer individuals are less than about 0.3. In certain embodiments, the DELFI scores for stage I cancer are between about 0.3 to less than 0.5. In certain embodiments, the DELFI scores for stage II cancer are between about 0.5 to less than 0.8. In certain embodiments, the DELFI scores for stage III cancer are between about 0.8 to less than 0.99. In certain embodiments, the DELFI scores for stage IV cancer are about 0.99 or greater. In certain embodiments, the DELFI score for stage I cancer is about 0.35. In certain embodiments, the DELFI score for stage II cancer is about 0.75. In certain embodiments, the DELFI score for stage III cancer is about 0.9. In certain embodiments, the DELFI score for stage IV cancer is about 0.99.

In certain embodiments, a method of diagnostically distinguishing between subjects with small cell lung cancer (SCLC) from those with non-small cell lung cancer (NSCLC) or without cancer, comprises: comparing differential expression of transcription factors in biological samples of SCLC, NSCLC or white blood cells; selecting at least one or more transcription factors having a higher differential expression as compared to the expression of transcription factors identified in the biological samples; extracting cell free (cfDNA) from the subject's biological sample; obtaining genome-wide fragmentation profiles of the cfDNA obtained from the subject to identify the at least one or more transcription factor binding sites; evaluating cfDNA coverage of the at least one or more transcription factor binding sites to determine fragment coverage and size as compared to non-cancer subjects or NSCLC subjects; thereby, diagnostically distinguishing between subjects with small cell lung cancer (SCLC) from those with non-small cell lung cancer (NSCLC) or without cancer.

In certain embodiments, the at least one transcription factor is Achaete-Scute Family basic helix-loop-helix Transcription Factor 1 (ASCL1).

In certain embodiments, the cfDNA fragment sizes in nucleic acid sequences comprising ASCL1 binding sites are larger in SCLC patients as compared to patients with NSCLC or non-cancer subjects.

In certain embodiments, the aggregate fragment coverage in nucleic acid sequences comprising ASCL1 binding sites is decreased in SCLC patients as compared to patients with NSCLC or non-cancer subjects.

In certain embodiments, a method of diagnostically distinguishing between subjects with small cell lung cancer (SCLC) from those with non-small cell lung cancer (NSCLC) or without cancer, the method comprises: extracting cell free (cfDNA) from the subject's biological sample; evaluating cfDNA coverage of Achaete-Scute Family basic helix-loop-helix Transcription Factor 1 (ASCL1) binding sites to determine fragment coverage and size as compared to non-cancer subjects or NSCLC subjects; thereby, diagnostically distinguishing between subjects with small cell lung cancer (SCLC) from those with non-small cell lung cancer (NSCLC) or without cancer.

In certain embodiments, the cfDNA fragment sizes in nucleic acid sequences comprising ASCL1 binding sites are larger in SCLC patients as compared to patients with NSCLC or non-cancer subjects.

In certain embodiments, the aggregate fragment coverage in nucleic acid sequences comprising ASCL1 binding sites is decreased in SCLC patients as compared to patients with NSCLC or non-cancer subjects.

In certain embodiments, the subject's fragmentation profile provides cell type-specific genome-wide transcription factor binding and is diagnostic of the type of lung cancer and histological subtypes.

In certain embodiments, the subject is administered cancer therapies.

In certain embodiments, the method of determining recurrence of cancer in a subject comprises the methods embodied herein.

In certain embodiments, the method of correcting GC content of a genome-wide fragmentation analyses, comprises: sequencing of whole genome libraries of cancer subjects and cancer-free subjects from samples not subjected to polymerase chain reaction (PCR) and samples subjected to a variable number of PCR cycles, filtering of adapter sequences, aligning sequence reads against a human reference genome and removing of duplicate reads, converting each aligned pair to a genomic interval, wherein the genomic interval represents sequenced DNA fragments, and selecting reads having a mapq score of at least 30 or greater.

In certain embodiments, the method further comprises tiling the reference genome into about 100-600 non-overlapping 1-10 Mb bins spanning about 1-3 GB of the genome thereby capturing large-scale epigenetic differences in fragmentation across the genome from low-coverage whole genome sequencing.

In certain embodiments, the ratios of the number of short to long (151 to 220 bp) fragments across the 100-600 non-overlapping 1-10 Mb bins were normalized for GC-content and library size.

In certain embodiments, the method further comprises obtaining the total number of fragments within each GC stratum comprises assigning of fragments to one of about 100 possible GC strata between 0 and 1.

In certain embodiments, the 1 indicates a fragment with all G and C nucleotides.

In certain embodiments, the method further comprises obtaining a distribution of fragment counts by GC stratum for non-cancer samples and the median of target distributions.

In certain embodiments, the, normalizing of sample-to-sample PCR biases in subjects comprises, assigning a weight wᵢ to the fragments in GC stratum i such that Σᵢ₌₁^{N}^{ᵢ}wᵢ=tᵢ where tᵢ denotes the number of fragments in the target distribution, N_i the total number of fragments in stratum i, and i=1, . . . 100. In certain embodiments, the normalizing of sample-to-sample variation in GC-biases and differences in library size comprises computing of GC-adjusted number of short and long fragments for each bin as the sum of the weights for the fragments aligned to that bin.

In certain embodiments, the fragmentation profiles are consistent among non-cancer subjects and subjects with non-malignant lung cancer.

In certain embodiments, the cancer subjects display widespread genome-wide variation.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The terms “aligned”, “alignment”, “mapped” or “aligning”, “mapping” refer to one or more sequences that are identified as a match in terms of the order of their nucleic acid molecules to a known sequence from a reference genome. Such alignment can be done manually or by a computer algorithm, examples including the Efficient Local Alignment of Nucleotide Data (ELAND) computer program distributed as part of the Illumina Genomics Analysts pipeline. The matching of a sequence read in aligning can be a 100% sequence match or less than 100% (non-perfect match).

The term “alternative allele” or “ALT” refers to an allele having one or more mutations relative to a reference allele, e.g., corresponding to a known gene.

The term “biomarker” means a distinctive biological or biologically derived indicator of a process, event or condition. Biomarkers can be used in methods of diagnosis, e.g. clinical screening, and prognosis assessment; and in monitoring the results of therapy, for identifying patients most likely to respond to a particular therapeutic treatment, as well as in drug screening and development. Biomarkers and uses thereof are valuable for identification of new drug treatments and for discovery of new targets for drug treatment. As used herein, the term “biomarker” refers to a molecule that is associated either quantitatively or qualitatively with a biological change. Examples of biomarkers include polypeptides, proteins or fragments of a polypeptide or protein; and polynucleotides, such as a gene product, RNA or RNA fragment; and other body metabolites. In certain embodiments, a “biomarker” means a compound that is differentially present (i.e., increased or decreased) in a biological sample from a subject or a group of subjects having a first phenotype (e.g., having a disease or condition) as compared to a biological sample from a subject or group of subjects having a second phenotype (e.g., not having the disease or condition or having a less severe version of the disease or condition). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using, for example, either Welch's T-test or Wilcoxon's rank-sum Test).

In addition, the term “biomarker” also includes the isoforms and/or post-translationally modified forms of any of the foregoing. The present invention contemplates the detection, measurement, quantification, determination and the like of both unmodified and modified (e.g., glycosylation, citrullination, phosphorylation, oxidation or other post-translational modification) proteins/polypeptides/peptides. In certain embodiments, it is understood that reference to the detection, measurement, determination, and the like, of a biomarker refers detection of the protein/polypeptide/peptide (modified and/or unmodified).

The term “cancer” as used herein is meant, a disease, condition, trait, genotype or phenotype characterized by unregulated cell growth or replication as is known in the art; including lung cancer (including non-small cell lung carcinoma), gastric cancer, colorectal cancer, as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, bladder cancer, prostate cancer, pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis.

The term “candidate variant,” “called variant,” or “putative variant” refers to one or more detected nucleotide variants of a nucleotide sequence, for example, at a position in the genome that is determined to be mutated. Generally, a nucleotide base is deemed a called variant based on the presence of an alternative allele on sequence reads obtained from a sample, where the sequence reads each cross over the position in the genome. The source of a candidate variant may initially be unknown or uncertain. During processing, candidate variants may be associated with an expected source such as genomic DNA (e.g., blood-derived) or cells impacted by cancer (e.g., tumor-derived). Additionally, candidate variants may be called as true positives. A variant of interest is particular variant of a genetic sequence that is to be measured, qualified, quantified, or detected. In some implementations, a variant of interest is a variant known or suspected to be associated with a condition, such as a cancer, a tumor, or a genetic disorder.

The term “cell free nucleic acid,” “cell free DNA,” or “cfDNA” refers to nucleic acid fragments that circulate in an individual's body (e.g., bloodstream) and originate from one or more healthy cells and/or from one or more cancer cells. Additionally cfDNA may come from other sources such as viruses, fetuses, etc.

The term “circulating tumor DNA” or “ctDNA” refers to nucleic acid fragments that originate from tumor cells or other types of cancer cells, which may be released into an individual's bloodstream as result of biological processes such as apoptosis or necrosis of dying cells or actively released by viable tumor cells.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

An “effective amount” as used herein, means an amount which provides a therapeutic or prophylactic benefit.

As used herein, the terms “fragmentation profile,” “position dependent differences in fragmentation patterns,” and “differences in fragment size and coverage in a position dependent manner across the genome” are equivalent and can be used interchangeably. As used herein, the terms “fragmentation profile,” “position dependent differences in fragmentation patterns,” and “differences in fragment size and coverage in a position dependent manner across the genome” are equivalent and can be used interchangeably. In some embodiments, determining a cfDNA fragmentation profile in a mammal can be used for identifying a mammal as having cancer. For example, cfDNA fragments obtained from a mammal (e.g., from a sample obtained from a mammal) can be subjected to low coverage whole-genome sequencing, and the sequenced fragments can be mapped to the genome (e.g., in non-overlapping windows) and assessed to determine a cfDNA fragmentation profile. As described herein, a cfDNA fragmentation profile of a mammal having cancer is more heterogeneous (e.g., in fragment lengths) than a cfDNA fragmentation profile of a healthy mammal (e.g., a mammal not having cancer). As such, this disclosure also provides methods and materials for assessing, monitoring, and/or treating mammals (e.g., humans) having, or suspected of having, cancer. In some embodiments, this document provides methods and materials for identifying a mammal as having cancer. For example, a sample (e.g., a blood sample) obtained from a mammal can be assessed to determine the presence and, optionally, the tissue of origin of the cancer in the mammal based, at least in part, on the cfDNA fragmentation profile of the mammal. In some embodiments, methods and materials for monitoring a mammal as having cancer are provided. For example, a sample (e.g., a blood sample) obtained from a mammal can be assessed to determine the presence of the cancer in the mammal based, at least in part, on the cfDNA fragmentation profile of the mammal. In some embodiments, methods and materials for identifying a mammal as having cancer, and administering one or more cancer treatments to the mammal to treat the mammal are provided. For example, a sample (e.g., a blood sample) obtained from a mammal can be assessed to determine if the mammal has cancer based, at least in part, on the cfDNA fragmentation profile of the mammal, and one or more cancer treatments can be administered to the mammal.

The term “genomic nucleic acid,” or “genomic DNA,” refers to nucleic acid including chromosomal DNA that originates from one or more healthy (e.g., non-tumor) cells. In various embodiments, genomic DNA can be extracted from a cell derived from a blood cell lineage, such as a white blood cell (WBC).

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some embodiments, the methods of the invention find use in experimental animals, in veterinary application, and in the development of animal models for disease, including, but not limited to, rodents including mice, rats, and hamsters, and primates.

The term “reference genome” as used herein may refer to a digital or previously identified nucleic acid sequence database, assembled as a representative example of a species or subject. Reference genomes may be assembled from the nucleic acid sequences from multiple subjects, sample or organisms and does not necessarily represent the nucleic acid makeup of a single person. Reference genomes may be used to for mapping of sequencing reads from a sample to chromosomal positions. For example, a reference genome used for human subjects as well as many other organisms is found at the National Center for Biotechnology Information at ncbi.nlm.nih.gov.

The term “read segment” or “read” refers to any nucleotide sequences including sequence reads obtained from an individual and/or nucleotide sequences derived from the initial sequence read from a sample obtained from an individual.

The terms “sample,” “patient sample,” “biological sample,” and the like, encompass a variety of sample types obtained from a patient, individual, or subject and can be used in a diagnostic, prognostic and/or monitoring assay. The patient sample may be obtained from a healthy subject, a diseased patient, or a patient with lung cancer. In certain embodiments, a sample that is “provided” can be obtained by the person (or machine) conducting the assay, or it can have been obtained by another, and transferred to the person (or machine) carrying out the assay. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiment, a sample comprises cerebrospinal fluid. In a specific embodiment, a sample comprises a blood sample. In another embodiment, a sample comprises a plasma sample. In yet another embodiment, a serum sample is used. The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

The term “sequence reads” refers to nucleotide sequences read from a sample obtained from an individual. Sequence reads can be obtained through various methods known in the art.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a schematic representation of DNA fragmentation and release from apoptotic lung cancer cells and white blood cells (WBCs). Nucleosomal DNA with variable length of linker DNA is preserved in the circulation with cancer cell cfDNA fragments having a more aberrant profile compared to the cfDNA fragments arising from the WBCs. Mapping of the cfDNA fragments along the genome and calculating the ratio of short fragments to long fragments reveals distinct patterns in cancer patients from the non-cancer individuals. FIG. 1B is a diagrammatic representation of the overall approach. Outline of the DELFI approach for early detection of lung cancer. A total of 365 patients from the LUCAS diagnostic cohort were used to derive genome-wide fragmentation profiles that were used to train and evaluate the diagnostic performance in this cohort using a cross validated machine learning model. A fixed model was used to validate the performance in an independent cohort of 46 lung cancer patients and 385 non-cancer individuals.

FIGS. 2A, 2B and 2C are a graphical read-out and heatmaps showing the cell-free DNA fragmentation profiles of lung cancer patients and non-cancer individuals. FIG. 2A: The ratio of short to long cfDNA fragments across the genome in 5 Mb bins was evaluated in plasma samples of lung cancer and non-cancer individuals from the LUCAS cohort. The non-cancer patients had similar fragmentation profiles compared to lung cancer patients that exhibited significant variation. FIG. 2B: Heatmap representation of the deviation of cfDNA fragmentation features across the genome for patients with lung cancer or non-cancer individuals compared to the mean of non-cancer individuals. Overall DELFI score and clinical characteristics are indicated to the left of the fragmentation deviation heatmap. FIG. 2C: Heatmap representation of principal component eigenvalues of the fragmentation profile features. The relative importance of the features are shown at the top (fragmentation changes) and right (chromosomal arm changes) of the heatmap, with colors indicating increases (red) or decreases (blue) of the coefficient of cancer risk. TCGA derived observations of chromosomal arm gains (red) and losses (blue) in lung adenocarcinoma (n=518) and squamous cell cancers (n=501) are indicated at the right margin. Agreement between the color of the variable importance bar in LUCAS and the TCGA copy number data indicates a correspondence between higher cancer risk due to decreases (blue) or increases (red) in chromosomal arm level representation in LUCAS and copy number amplifications (red) and copy number deletions (blue) in TCGA, respectively.

FIGS. 3A-3C are a series of plots demonstrating the performance of DELFI analyses for lung cancer patients and non-cancer individuals. FIG. 3A: DELFI score distribution across non-cancer individuals and cancer patients, stratified by stage and histology groups in the LUCAS cohort. The box-plot shows the median DELFI score and the inter-quartile range with the individual sample values overlaid as dots. The non-cancer cases with or without benign lesions have a lower DELFI score compared to cancer cases and there is a stepwise increase in DELFI score by stage. The highest median DELFI score is observed in SCLC cases. The dotted vertical line in the ROC figures represents an 80% specificity as a decision boundary FIG. 3B: ROC analyses of the overall LUCAS cohort as well as by stage and histology. FIG. 3C: Analysis of a DELFI fixed model and score cutoff of 0.344 determined from the LUCAS cohort was applied in the validation cohort. The performance of this classifier in the independent cohort was similar to LUCAS in both specificity (left) and sensitivity (right) across all tumor stages. The number of samples in the training and validation sets are indicated in the labels of the horizontal axis. The intervals presented reflect a 90% confidence interval.

FIGS. 4A-4C are a series of graphs demonstrating the relationship of size and invasiveness of lung cancer with DELFI score. FIG. 4A: Boxplot graph of the DELFI scores of non-metastatic patients with lung cancer categorized by T stage or N stage in the LUCAS cohort. An incremental increase of the DELFI score by T stage from T1-T4 was observed (p<0.01, Kruskall-Wallis, df=3). Lung cancer patients without involvement of lymph nodes had a significantly lower DELFI scores compared to patients with nodal spread (Wilcoxon rank sum test, p<0.001). FIG. 4B: The stepwise increase in DELFI score by T and N stage was maintained when considering both T and N stages in each patient (Kruskall-Wallis, df=6, p<0.01). FIG. 4C: Patients with primary lung cancer were stratified in two groups based on a DELFI cutoff of 0.5 (n=93). Patients with a DELFI score>0.5 had a significantly worse cancer specific survival compared to patients with DELFI score<0.5.

FIGS. 5A-5F are a series plots, a box graph and an unsupervised clustering analysis demonstrating that the genome-wide fragmentation profiles can distinguish SCLC from NSCLC. FIG. 5A: Expression of ASCL1 transcription factor in TCGA RNA-seq analyses of SCLCs (n=79) is high compared to NSCLC (n=1046) or WBC (755) samples. FIG. 5B:Unsupervised clustering analyses of gene expression in TCGA lung cancer cohorts shows that genes with ASCL1 binding sites are differentially expressed between SCLCs and NSCLCs. Genome-wide cfDNA fragmentation analyses at ASCL1 binding sites in LUCAS cohort patients reveals a decrease in coverage near transcription factor binding sites of SCLC patients compared to non-cancer individuals (FIG. 5C) or DELFI positive patients with SCLC compared to other individuals (FIG. 5E). These molecular features can distinguish SCLC patients (n=11) from non-cancer individuals (n=158) (FIG. 5D, AUC=0.92) and DELFI positive SCLC patients (n=10) from NSCLC patients and others (n=115)(FIG. 5F, AUC=0.98), with high accuracy.

FIGS. 6A-6G are a series of graphs and a schematic representation demonstrating modeling the implementation of DELFI in lung cancer screening. FIG. 6A: Schematic representation of current clinical practice for lung cancer screening (top) and the proposed approach in combination with the DELFI test (bottom). In the combined approach, individuals at high-risk for lung cancer would undergo an annual blood draw that would be scored by the DELFI test, and individuals with a positive result would subsequently undergo an LDCT scan and subsequent diagnostic evaluation for detection of lung cancer, while individuals with a DELFI negative result would repeat their screening annually. FIG. 6B: Sensitivity of DELFI alone, or DELFI followed by LDCT for lung cancer detection was determined assuming a specificity of 80% for the single or combined analyses. For these analyses, we considered individuals with lung cancer as those detected at baseline with LDCT, although three individuals were identified with lung cancer at a repeat LDCT within a year. The number of individuals in the LUCAS cohort are as follows: stage I n=15, II n=7, III n=35, IV n=72; and individuals in the cohort with lung adenocarcinoma comprised stage I n=8, II n=3, III n=14, IV n=37. The number of individuals are indicated schematically by the size of the dots and in Table 1. FIG. 6C: The uncertainty of sensitivity and specificity of LDCT alone, as well as DELFI were modeled followed by LDCT for screening in a theoretical population of 100,000 high-risk individuals. Predictive distributions for the number of lung cancers detected (FIG. 6D), accuracy (FIG. 6E), rate of unnecessary procedures (FIG. 6F), and positive predictive values (FIG. 6G) among these individuals incorporated variation in both the prevalence of lung cancer and adherence to image- and blood-based screening.

FIG. 7 is a schematic of a diagnostic algorithm for the samples analyzed from the LUCAS diagnostic cohort. Patients in the LUCAS cohort were referred for a diagnostic workup after a positive finding on a chest X-ray or chest CT. All patients received a plasma and serum blood draw at the time of clinic visit as well as a chest CT to confirm the original imaging finding. The patients were stratified into low suspicion and high suspicion for lung cancer groups. Patients with low suspicion (n=150) were followed clinically and radiographically for up to 7 years. 145 of these patients had not developed lung cancer at the time of last follow-up whereas 5 of these patients had lung cancer diagnosed based on clinical history and imaging characteristics and were confirmed to have lung cancer on autopsy. Patients with high suspicion (n=208) underwent additional workup including PET-CT scan and a lung biopsy. 87 of these patients were diagnosed with a non-malignant nodule whereas 124 patients had a histologically confirmed lung cancer by biopsy.

FIGS. 8A-8C are a series of plots showing the fragmentation profiles in matched DNase digested lymphocyte DNA with various cycles of PCR amplification. FIG. 8A: Standardized fragment coverage of the same sample without amplification (0 cycles PCR, n=1) compared to technical replicates of 4 cycles of PCR (n=4) and 12 cycles of PCR (n=3). There were minimal effects of 4 cycles of PCR compared to no amplification, while 12 cycles were visibly different. FIG. 8B: Principal component analyses of fragmentation profiles without GC correction highlights the similarity of 0 and 4 cycle PCR fragmentation profiles.

FIG. 8C: Fragment-level GC correction of sequences from 4 or 12 cycle PCR libraries were similar to the naturally occurring fragmentation profiles without amplification, while bin-level GC correction only partially alleviated GC bias in 4 or 12 cycle PCR libraries.

FIG. 9 is a heatmap representation of the variation of feature contributions to the final DELFI model over 50 training iterations. The heatmap indicates the scaled regression coefficients of each model feature (vertical axis) across the 50 training sets (five-fold×10 repeats, horizontal axis). The gray boxes indicate models that do not utilize the indicated principal components (PC). The right margin represents the final model used for external validation using the available data from the LUCAS analyses.

FIGS. 10A-10D are a series of graphs showing the effect of smoking status, age, and comorbidities on DELFI scores in non-cancer individuals. FIG. 10A: The DELFI score in individuals without cancer was similar for those currently smoking versus never smokers or prior smokers (Kruskal-Wallis test, df=154, p=0.47), as well as across pack-year groups (Kruskal-Wallis test, df=154, p=0.47). FIGS. 10B, 10C: Individuals without cancer and a diagnosis of autoimmune diseases or COPD had similar DELFI scores to those individuals without these conditions (p=0.26, p=0.37 respectively). FIG. 10D: DELFI scores were not different among clinically relevant age groups (Kruskal-Wallis, df=2, p=0.18).

FIG. 11 is a series of plots showing DELFI scores and serum protein markers in non-cancer individuals. Correlation analyses of the DELFI score (vertical axes) with serum protein levels (horizontal axes) in healthy individuals. CRP and YKL-40 levels had weak correlations with DELFI scores (Spearman correlation coefficients: 0.17, p=0.04; 0.12, p=0.02, respectively). There was no correlation between IL-6 (p=0.08) or CEA (p=0.5) and DELFI scores.

FIGS. 12A-12C are a series of graphs showing a comparison of performance of DELFI approach with other genomic approaches. FIG. 12A: Implementation of the model features and GC bias correction described in the Cristiano et al. manuscript 23 in the LUCAS cohort shows a similar performance compared to the model implemented herein although the current DELFI model has higher sensitivity at high specificity ranges. FIG. 12B: The current DELFI model outperforms ichor analyses or assessment of median fragment lengths. The dotted vertical lines in the ROC figures represent 95%, 90% and 80% specificities. FIG. 12C: Comparison of median fragment sizes by cohort shows similar median fragment lengths for both the LUCAS and the validation cohort for non-cancer individuals as well as patients with cancer separated by stage.

FIG. 13 is a series of graphs demonstrating the performance of DELFI in the lung cancer validation cohort. Analysis of the validation cohort comprising 385 non-cancer individuals and 46 lung cancer patients revealed performance metrics similar to the LUCAS cohort across different stages and histological subtypes. The graphs show the specificity and sensitivity of DELFI when a fixed cutoff is used for both the LUCAS and the validation cohort. Each row represents a different DELFI score cutoff (0.252, 0.303, 0.344, or 0.377) that corresponds to a 70%, 75%, 80% or 85% specificity, respectively, as indicated from top to bottom. The intervals presented in the figure reflect a 90% confidence interval.

FIG. 14 is a graph showing the CEA levels across non-cancer individuals and lung cancer patients. Distribution of serum CEA levels (vertical axes) among diagnostic groups, stages, and histological subtypes.

FIGS. 15A and 15B are a series of graphs demonstrating the performance of DELFIₘᵤₗₜᵢ analyses for lung cancer patients and non-cancer individuals. FIG. 15A: DELFIₘᵤₗₜᵢ score distribution across stages and histological subtypes. FIG. 15B: DELFI ROC curves by stage and histology. The dotted vertical lines in the ROC figures represent 95%, 90% and 80% specificities.

FIGS. 16A-16D are a series of graphs demonstrating that cfDNA fragment sizes at ASCL1 binding sites can distinguish SCLC from non-cancer individuals and NSCLC patients. Genome-wide cfDNA fragmentation analyses at ASCL1 binding sites in LUCAS cohort patients reveals an increase in fragment sizes near transcription factor binding sites of SCLC patients compared to non-cancer individuals (FIG. 16A) or individuals with other cancers (FIG. 16C). This molecular features can distinguish SCLC patients from non-cancer individuals (FIG. 16B, AUC=0.91) and SCLC from NSCLC patients (FIG. 16D, AUC=0.94), with high accuracy.

FIGS. 17A and 17B are graphs showing the DELFI score and clinical outcome in lung cancer patients. FIG. 17A: Patients with stage IV primary lung cancer with DELFI score <0.5 revealed a significantly longer cancer-specific survival compared to patients with DELFI scores >0.5. FIG. 17B: To assess whether the DELFI score was an independent prognostic factor of cancer-specific overall survival we calculated the Cox proportional hazard ratios with high or low DELFI scores, histologic groups, and stage as covariates. Patients with DELFI scores >0.5 had a HR of 2.53 compared to patients with DELFI scores <0.5 (p<0.001) after adjusting for histologic group and stage. The intervals indicate a 95% confidence interval for the hazards ratio.

FIGS. 18A and 18B are graphs demonstrating that DELFI can identify molecular recurrence prior to clinical recurrence. FIG. 18A: Among patients with prior history of cancer, the ones that developed tumor recurrence during the follow up period had a significantly higher DELFI score compared to the ones that developed no recurrence (p=0.004). FIG. 18B: Patients with prior history of cancer, no evidence of cancer on baseline assessment and a positive DELFI score had a significantly shorter progression-free survival compared to ones with a negative DELFI score (p<0.01).

### DETAILED DESCRIPTION

There is an urgent unmet clinical need for the development of non-invasive approaches to improve standard of care cancer screening that can increase accessibility among high-risk individuals and ultimately the general population. Biomarker development for the early detection of lung cancer has potential clinical applications in screening as well as for discriminating malignancy in round opacities identified as nodules on chest imaging studies⁸. Investigation of proteins⁹⁻¹¹, autoantibodies¹², gene expression profiles¹³ and microRNAs¹⁴ in the blood or airway epithelium have yielded promising biomarker candidates for early detection of lung cancer although some may be affected by age as well as systemic inflammation induced by prolonged exposure to smoking and other conditions, and none are yet approved for clinical use¹⁴.

The rapid technological and analytical advancements in liquid biopsy analyses have identified cancer-related features in the cfDNA compartment of blood and provided a new avenue for early detection of cancer. Mutations in circulating tumor DNA (ctDNA) can be directly detected in early stage lung cancer patients without prior knowledge of these alterations in tumors¹⁵⁻¹⁸. However, given the relatively small number of sequence alterations that can be assessed by targeted high coverage sequencing, many individuals with cancer may be missed by such approaches and may also require sequencing of white blood cells (WBCs) to eliminate changes that result from clonal hematopoiesis^{16,17,19}. To increase the sensitivity of detection of early stage cancers, a genome-wide approach was developed using machine learning for analysis of cfDNA fragmentation profiles called DELFI (DNA evaluation of fragments for early interception)²⁰. This multi-feature analysis permits evaluation of millions of cfDNA fragments simultaneously, increasing number of tumor-derived epigenomic and genomic changes that can be detected. In this study, the methodology was improved and applied to or lung cancer detection in a prospectively collected diagnostic cohort comprising patients with lung cancer as well as non-cancer individuals. It is also disclosed herein, the evaluation of the combining this methodology with plasma protein biomarkers and blood cell counts, thereby examining genomic, epigenomic, protein, and cellular features for early cancer detection. Through this effort, a clinical framework is provided by which a non-invasive liquid biopsy approach could be incorporated in the clinic, combining the DELFI with other markers and low dose helical computed tomography (LDCT) for early lung cancer detection.

### DNA Evaluation of Fragments for Early Interception (DELFI)

DNA Evaluation of Fragments for early Interception (DELFI) was previously developed, Cristiano S, Leal A, Phallen J, et al. Genome-wide cell-free DNA fragmentation in patients with cancer. Nature 2019; 570:385-9 incorporated herein in its entirety, and used to evaluate genome-wide fragmentation patterns of cfDNA of 236 patients with breast, colorectal, lung, ovarian, pancreatic, gastric, or bile duct cancers as well as 245 healthy individuals. These analyses revealed that cfDNA profiles of healthy individuals reflected nucleosomal fragmentation patterns of white blood cells, while patients with cancer had altered fragmentation profiles. DELFI had sensitivities of detection ranging from 57% to >99% among the seven cancer types at 98% specificity and identified the tissue of origin of the cancers to a limited number of sites in 75% of embodiments. Assessing cfDNA (e.g., using DELFI) provide a screening approach for early detection of cancer, which can increase the chance for successful treatment of a patient having cancer. Assessing cfDNA (e.g., using DELFI) can also provide an approach for monitoring cancer, which can increase the chance for successful treatment and improved outcome of a patient having cancer. In addition, a cfDNA fragmentation profile can be obtained from limited amounts of cfDNA and using inexpensive reagents and/or instruments.

Accordingly, in certain embodiments, a method of diagnosing cancer in a subject, comprises extracting cell free (cfDNA) from the subject's biological sample; generating genomic libraries from the extracted cfDNA and whole genome sequencing of cfDNA fragments; mapping of the cfDNA fragments to a genomic origin and evaluating fragment length and obtaining genome-wide fragmentation profiles for each sample; identifying protein biomarkers of the subject; comparing the subject's cfDNA fragmentation profile and protein biomarkers with normal reference non-cancer subjects. In certain embodiments, the cancer is lung cancer.

In certain embodiments, the method further comprises subjecting the subject to a low dose helical computed tomography (LDCT). In certain embodiments, the method further comprises comparing clinical data between the subject diagnosed as having lung cancer and normal non-cancer subjects. In certain embodiments, the cfDNA fragment mean length and profiles are similar among non-cancer individuals. In certain embodiments, the cfDNA fragment profiles of cancer subjects vary. In certain embodiments, the serum levels of or one or more tumor antigens, cytokines or proteins are measured.

In certain embodiments a DELFI score is generated, wherein the principle component analysis is incorporated into a machine learning predictive model to generate a score for each subject as an average over cross-validation repeats (DELFI score(s)). In certain embodiments, the DELFI scores for non-cancer individuals are less than about 0.3. In certain embodiments, the DELFI scores for stage I cancer are between about 0.3 to less than 0.5. In certain embodiments, the DELFI scores for stage II cancer are between about 0.5 to less than 0.8. In certain embodiments, the DELFI scores for stage III cancer are between about 0.8 to less than 0.99. In certain embodiments, the DELFI scores for stage IV cancer are about 0.99 or greater. In certain embodiments, the DELFI score for stage I cancer is about 0.35. In certain embodiments, the DELFI score for stage II cancer is about 0.75. In certain embodiments, the DELFI score for stage III cancer is about 0.9. In certain embodiments, the DELFI score for stage IV cancer is about 0.99.

cfDNA Fragmentation Profiles: A cfDNA fragmentation profile can include one or more cfDNA fragmentation patterns. A cfDNA fragmentation pattern can include any appropriate cfDNA fragmentation pattern. Examples of cfDNA fragmentation patterns include, without limitation, median fragment size, fragment size distribution, ratio of small cfDNA fragments to large cfDNA fragments, and the coverage of cfDNA fragments. In some embodiments, a cfDNA fragmentation pattern includes two or more (e.g., two, three, or four) of median fragment size, fragment size distribution, ratio of small cfDNA fragments to large cfDNA fragments, and the coverage of cfDNA fragments. In some embodiments, cfDNA fragmentation profile can be a genome-wide cfDNA profile (e.g., a genome-wide cfDNA profile in windows across the genome). In some embodiments, cfDNA fragmentation profile can be a targeted region profile. A targeted region can be any appropriate portion of the genome (e.g., a chromosomal region). Examples of chromosomal regions for which a cfDNA fragmentation profile can be determined as described herein include, without limitation, a portion of a chromosome (e.g., a portion of 2q, 4p, 5p, 6q, 7p, 8q, 9q, 10q, 11q, 12q, and/or 14q) and a chromosomal arm (e.g., a chromosomal arm of 8q, 13q, 11q, and/or 3p). In some embodiments, a cfDNA fragmentation profile can include two or more targeted region profiles.

In some embodiments, a cfDNA fragmentation profile can be used to identify changes (e.g., alterations) in cfDNA fragment lengths. An alteration can be a genome-wide alteration or an alteration in one or more targeted regions/loci. A target region can be any region containing one or more cancer-specific alterations. In some embodiments, a cfDNA fragmentation profile can be used to identify (e.g., simultaneously identify) from about 10 alterations to about 500 alterations (e.g., from about 25 to about 500, from about 50 to about 500, from about 100 to about 500, from about 200 to about 500, from about 300 to about 500, from about 10 to about 400, from about 10 to about 300, from about 10 to about 200, from about 10 to about 100, from about 10 to about 50, from about 20 to about 400, from about 30 to about 300, from about 40 to about 200, from about 50 to about 100, from about 20 to about 100, from about 25 to about 75, from about 50 to about 250, or from about 100 to about 200, alterations).

In some embodiments, a cfDNA fragmentation profile can be used to detect tumor-derived DNA. For example, a cfDNA fragmentation profile can be used to detect tumor-derived DNA by comparing a cfDNA fragmentation profile of a mammal having, or suspected of having, cancer to a reference cfDNA fragmentation profile (e.g., a cfDNA fragmentation profile of a healthy mammal and/or a nucleosomal DNA fragmentation profile of healthy cells from the mammal having, or suspected of having, cancer). In some embodiments, a reference cfDNA fragmentation profile is a previously generated profile from a healthy mammal. For example, methods provided herein can be used to determine a reference cfDNA fragmentation profile in a healthy mammal, and that reference cfDNA fragmentation profile can be stored (e.g., in a computer or other electronic storage medium) for future comparison to a test cfDNA fragmentation profile in mammal having, or suspected of having, cancer. In some embodiments, a reference cfDNA fragmentation profile (e.g., a stored cfDNA fragmentation profile) of a healthy mammal is determined over the whole genome. In some embodiments, a reference cfDNA fragmentation profile (e.g., a stored cfDNA fragmentation profile) of a healthy mammal is determined over a subgenomic interval.

In some embodiments, a cfDNA fragmentation profile can be used to identify a mammal (e.g., a human) as having cancer (e.g., a colorectal cancer, a lung cancer, a breast cancer, a gastric cancer, a pancreatic cancer, a bile duct cancer, and/or an ovarian cancer).

A cfDNA fragmentation profile can include a cfDNA fragment size pattern. cfDNA fragments can be any appropriate size. For example, cfDNA fragment can be from about 50 base pairs (bp) to about 400 bp in length. As described herein, a mammal having cancer can have a cfDNA fragment size pattern that contains a shorter median cfDNA fragment size than the median cfDNA fragment size in a healthy mammal A healthy mammal (e.g., a mammal not having cancer) can have cfDNA fragment sizes having a median cfDNA fragment size from about 166.6 bp to about 167.2 bp (e.g., about 166.9 bp). In some embodiments, a mammal having cancer can have cfDNA fragment sizes that are, on average, about 1.28 bp to about 2.49 bp (e.g., about 1.88 bp) shorter than cfDNA fragment sizes in a healthy mammal. For example, a mammal having cancer can have cfDNA fragment sizes having a median cfDNA fragment size of about 164.11 bp to about 165.92 bp (e.g., about 165.02 bp).

A cfDNA fragmentation profile can include a cfDNA fragment size distribution. As described herein, a mammal having cancer can have a cfDNA size distribution that is more variable than a cfDNA fragment size distribution in a healthy mammal. In some embodiments, a size distribution can be within a targeted region. A healthy mammal (e.g., a mammal not having cancer) can have a targeted region cfDNA fragment size distribution of about 1 or less than about 1. In some embodiments, a mammal having cancer can have a targeted region cfDNA fragment size distribution that is longer (e.g., 10, 15, 20, 25, 30, 35, 40, 45, 50 or more bp longer, or any number of base pairs between these numbers) than a targeted region cfDNA fragment size distribution in a healthy mammal. In some embodiments, a mammal having cancer can have a targeted region cfDNA fragment size distribution that is shorter (e.g., 10, 15, 20, 25, 30, 35, 40, 45, 50 or more bp shorter, or any number of base pairs between these numbers) than a targeted region cfDNA fragment size distribution in a healthy mammal. In some embodiments, a mammal having cancer can have a targeted region cfDNA fragment size distribution that is about 47 bp smaller to about 30 bp longer than a targeted region cfDNA fragment size distribution in a healthy mammal. In some embodiments, a mammal having cancer can have a targeted region cfDNA fragment size distribution of, on average, a 10, 11, 12, 13, 14, 15, 15, 17, 18, 19, 20 or more bp difference in lengths of cfDNA fragments. For example, a mammal having cancer can have a targeted region cfDNA fragment size distribution of, on average, about a 13 bp difference in lengths of cfDNA fragments. In some embodiments, a size distribution can be a genome-wide size distribution. A healthy mammal (e.g., a mammal not having cancer) can have very similar distributions of short and long cfDNA fragments genome-wide. In some embodiments, a mammal having cancer can have, genome-wide, one or more alterations (e.g., increases and decreases) in cfDNA fragment sizes. The one or more alterations can be any appropriate chromosomal region of the genome. For example, an alteration can be in a portion of a chromosome. Examples of portions of chromosomes that can contain one or more alterations in cfDNA fragment sizes include, without limitation, portions of 2q, 4p, 5p, 6q, 7p, 8q, 9q, 10q, 11q, 12q, and 14q. For example, an alteration can be across a chromosome arm (e.g., an entire chromosome arm).

A cfDNA fragmentation profile can include a ratio of small cfDNA fragments to large cfDNA fragments and a correlation of fragment ratios to reference fragment ratios. As used herein, with respect to ratios of small cfDNA fragments to large cfDNA fragments, a small cfDNA fragment can be from about 100 bp in length to about 150 bp in length. As used herein, with respect to ratios of small cfDNA fragments to large cfDNA fragments, a large cfDNA fragment can be from about 151 bp in length to 220 bp in length. As described herein, a mammal having cancer can have a correlation of fragment ratios (e.g., a correlation of cfDNA fragment ratios to reference DNA fragment ratios such as DNA fragment ratios from one or more healthy mammals) that is lower (e.g., 2-fold lower, 3-fold lower, 4-fold lower, 5-fold lower, 6-fold lower, 7-fold lower, 8-fold lower, 9-fold lower, 10-fold lower, or more) than in a healthy mammal. A healthy mammal (e.g., a mammal not having cancer) can have a correlation of fragment ratios (e.g., a correlation of cfDNA fragment ratios to reference DNA fragment ratios such as DNA fragment ratios from one or more healthy mammals) of about 1 (e.g., about 0.96). In some embodiments, a mammal having cancer can have a correlation of fragment ratios (e.g., a correlation of cfDNA fragment ratios to reference DNA fragment ratios such as DNA fragment ratios from one or more healthy mammals) that is, on average, about 0.19 to about 0.30 (e.g., about 0.25) lower than a correlation of fragment ratios (e.g., a correlation of cfDNA fragment ratios to reference DNA fragment ratios such as DNA fragment ratios from one or more healthy mammals) in a healthy mammal.

A cfDNA fragmentation profile can include coverage of all fragments. Coverage of all fragments can include windows (e.g., non-overlapping windows) of coverage. In some embodiments, coverage of all fragments can include windows of small fragments (e.g., fragments from about 100 bp to about 150 bp in length). In some embodiments, coverage of all fragments can include windows of large fragments (e.g., fragments from about 151 bp to about 220 bp in length).

A cfDNA fragmentation profile can be obtained using any appropriate method. In some embodiments, cfDNA from a mammal (e.g., a mammal having, or suspected of having, cancer) can be processed into sequencing libraries which can be subjected to whole genome sequencing (e.g., low-coverage whole genome sequencing), mapped to the genome, and analyzed to determine cfDNA fragment lengths. Mapped sequences can be analyzed in non-overlapping windows covering the genome. Windows can be any appropriate size. For example, windows can be from thousands to millions of bases in length. As one non-limiting example, a window can be about 5 megabases (Mb) long. Any appropriate number of windows can be mapped. For example, tens to thousands of windows can be mapped in the genome. For example, hundreds to thousands of windows can be mapped in the genome. A cfDNA fragmentation profile can be determined within each window.

In some embodiments, methods and materials described herein also can include machine learning. For example, machine learning can be used for identifying an altered fragmentation profile (e.g., using coverage of cfDNA fragments, fragment size of cfDNA fragments, coverage of chromosomes, and mtDNA).

### Biomarkers

In certain embodiments, detection of one or more biomarkers from patients are combined with DELFI as described in detail in the examples section which follows. In certain embodiments, the serum levels of or one or more tumor antigens, cytokines or proteins are measured, compared etc.

As used herein, the terms “comparing” or “comparison” refers to making an assessment of how the proportion, level or cellular localization of one or more biomarkers in a sample from a patient relates to the proportion, level or cellular localization of the corresponding one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in standard or control sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels/ratios that correspond to, for example, a patient having lung cancer, not having lung cancer, is responding to treatment for myocardial injury, is not responding to treatment for myocardial injury, is/is not likely to respond to a particular myocardial injury treatment, or having/not having another disease or condition. In a specific embodiment, the term “comparing” refers to assessing whether the level of one or more biomarkers of the present invention in a sample from a patient is the same as, more or less than, different from other otherwise correspond (or not) to levels/ratios of the same biomarkers in a control sample (e.g., predefined levels/ratios that correlate to healthy individuals, lung cancer levels/ratios, etc.). In another embodiment, the terms “comparing” or “comparison” refers to making an assessment of how the proportion, level or cellular localization of one or more biomarkers in a sample from a patient relates to the proportion, level or cellular localization of another biomarker in the same sample. For example, a ratio of one biomarker to another from the same patient sample can be compared. In another embodiment, a level of one biomarker in a sample (e.g., a post-translationally modified biomarker protein) can be compared to the level of the same biomarker (e.g., unmodified biomarker protein) in the sample. Ratios of modified:unmodified biomarker proteins can be compared to other protein ratios in the same sample or to predefined reference or control ratios.

In certain embodiments in which the relationship of the biomarkers are described in terms of a ratio, the ratio can include 1-fold, 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, 40-, 41-, 42-, 43-, 44-, 45-, 46-, 47-, 48-, 49-, 50-, 51-, 52-, 53-, 54-, 55-, 56-, 57-, 58-, 59-, 60-, 61-, 62-, 63-, 64-, 65-, 66-, 67-, 68-, 69-, 70-, 71-, 72-, 73-, 74-, 75-, 76-, 77-, 78-, 79-, 80-, 81-, 82-, 83-, 84-, 85-, 86-, 87-, 88-, 89-, 90-, 91-, 92-, 93-, 94-, 95-, 96-, 97-, 98-, 99-, 100-fold or more difference (higher or lower). Alternatively, the difference can include 0.9-fold, 0.8-fold, 0.7-fold, 0.7-fold, 0.6-fold, 0.5-fold, 0.4-fold, 0.3-fold, 0.2-fold, and 0.1-fold (higher or lower) depending on context. The foregoing can also be expressed in terms of a range (e.g., 1-5 fold/times higher or lower) or a threshold (e.g., at least 2-fold/times higher or lower).

In other embodiments, a particular set or pattern of the amounts of one or more biomarkers may be correlated to a patient being unaffected (i.e., indicates a patient does not have cancer, e.g. lung cancer). In certain embodiments, “indicating,” or “correlating,” as used according to the present disclosure, may be by any linear or non-linear method of quantifying the relationship between levels/ratios of biomarkers to a standard, control or comparative value for the assessment of the diagnosis, prediction of cancer or cancer progression, assessment of efficacy of clinical treatment, identification of a patient that may respond to a particular treatment regime or pharmaceutical agent, monitoring of the progress of treatment, and in the context of a screening assay, for the identification of an anti-cancer therapeutics.

In certain embodiments, the biomarkers detected are tumor antigens. In certain embodiments, the one or more tumor antigens comprise: carcinoembryonic antigen (CEA), CA19-9, CA 125, tissue polypeptideantigen (TSA), CYFRA-21-1, neuron-specific enolase, progastrin-releasing peptide (ProGRP), plasma kalikrein B1 (KLKB1), serum amyloid A, haptoglobin-alpha-2, ADAM-17, osteoprotegerin, pentraxin 3, follistatin, tumor necrosis factor receptor superfamily member 1A or combinations thereof.

In certain embodiments, the one or more proteins comprise C-reactive protein (CRP), Chitinase-3-like protein 1 (YKL-40/CHI3L1) or fragments thereof.

Other tumor antigens include (note, the cancer indications indicated represent non-limiting examples): aminopeptidase N (CD13), annexin Al, B7-H3 (CD276, various cancers), CA125 (ovarian cancers), CA15-3 (carcinomas), CA19-9 (carcinomas), L6 (carcinomas), Lewis Y (carcinomas), Lewis X (carcinomas), alpha fetoprotein (carcinomas), CA242 (colorectal cancers), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma, B-cell neoplasmas, autoimmune diseases), CD21 (B-cell lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (metastatic melanoma, sarcoma), CD52 (leukemia), CD56 (small cell lung cancers, ovarian cancer, Merkel cell carcinoma, and the liquid tumor, multiple myeloma), CD66e (carcinomas), CD70 (metastatic renal cell carcinoma and non-Hodgkin lymphoma), CD74 (multiple myeloma), CD80 (lymphoma), CD98 (carcinomas), CD123 (leukemia), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (CEA, CD66e) (breast, colorectal and lung cancers), DLL4 (A-like-4), EGFR (various cancers), CTLA4 (melanoma), CXCR4 (CD 184, heme-oncology, solid tumors), Endoglin (CD 105, solid tumors), EPCAM (epithelial cell adhesion molecule, bladder, head, neck, colon, NHL prostate, and ovarian cancers), ERBB2 (lung, breast, prostate cancers), FCGR1 (autoimmune diseases), FOLR (folate receptor, ovarian cancers), FGFR (carcinomas), GD2 ganglioside (carcinomas), G-28 (a cell surface antigen glycolipid, melanoma), GD3 idiotype (carcinomas), heat shock proteins (carcinomas), HER1 (lung, stomach cancers), HER2 (breast, lung and ovarian cancers), HLA-DR10 (NHL), HLA-DRB (NHL, B cell leukemia), human chorionic gonadotropin (carcinomas), IGF1R (solid tumors, blood cancers), IL-2 receptor (T-cell leukemia and lymphomas), IL-6R (multiple myeloma, RA, Castleman's disease, IL6 dependent tumors), integrins (αvβ3, α5β1, α6β4, α11β3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 (breast, ovarian, cervix, bronchus and gastrointestinal cancer), MUC16 (CA125) (ovarian cancers), CEA (colorectal cancer), gp100 (melanoma), MARTI (melanoma), MPG (melanoma), MS4A1 (membrane-spanning 4-domains subfamily A, small cell lung cancers, NHL), nucleolin, Neu oncogene product (carcinomas), P21 (carcinomas), nectin-4 (carcinomas), paratope of anti-(N-glycolylneuraminic acid, breast, melanoma cancers), PLAP-like testicular alkaline phosphatase (ovarian, testicular cancers), PSMA (prostate tumors), PSA (prostate), ROB04, TAG 72 (tumor associated glycoprotein 72, AML, gastric, colorectal, ovarian cancers), T cell transmembrane protein (cancers), Tie (CD202b), tissue factor, TNFRSF10B (tumor necrosis factor receptor superfamily member 10B, carcinomas), TNFRSF13B (tumor necrosis factor receptor superfamily member 13B, multiple myeloma, NHL, other cancers, RA and SLE), TPBG (trophoblast glycoprotein, renal cell carcinoma), TRAIL-R1 (tumor necrosis apoptosis inducing ligand receptor 1, lymphoma, NHL, colorectal, lung cancers), VCAM-1 (CD106, Melanoma), VEGF, VEGF-A, VEGF-2 (CD309) (various cancers). Some other tumor associated antigens have been reviewed (Gerber, et al, mAbs 2009 1:247-253; Novellino et al, Cancer Immunol Immunother. 2005 54:187-207, Franke, et al, Cancer Biother Radiopharm. 2000, 15:459-76, Guo, et al., Adv Cancer Res. 2013; 119: 421-475, Parmiani et al. J Immunol. 2007 178:1975-9). Examples of these antigens include Cluster of Differentiations (CD4, CD5, CD6, CD7, CD8, CD9, CD10, CD11a, CD11b, CD11c, CD12w, CD14, CD15, CD16, CDwl7, CD18, CD21, CD23, CD24, CD25, CD26, CD27, CD28, CD29, CD31, CD32, CD34, CD35, CD36, CD37, CD41, CD42, CD43, CD44, CD45, CD46, CD47, CD48, CD49b, CD49c, CD53, CD54, CD55, CD58, CD59, CD61, CD62E, CD62L, CD62P, CD63, CD68, CD69, CD71, CD72, CD79, CD81, CD82, CD83, CD86, CD87, CD88, CD89, CD90, CD91, CD95, CD96, CD100, CD103, CD105, CD106, CD109, CD117, CD120, CD127, CD133, CD134, CD135, CD138, CD141, CD142, CD143, CD144, CD147, CD151, CD152, CD154, CD156, CD158, CD163, CD166, .CD168, CD184, CDwl86, CD195, CD202 (a, b), CD209, CD235a, CD271, CD303, CD304), annexin Al, nucleolin, endoglin (CD105), ROB04, amino-peptidase N, -like-4 (DLL4), VEGFR-2 (CD309), CXCR4 (CD184), Tie2, B7-H3, WT1, MUC1, LMP2, HPV E6 E7, EGFRvIII, HER-2/neu, idiotype, MAGE A3, p53 nonmutant, NY-ESO-1, GD2, CEA, MelanA/MART1, Ras mutant, gp100, p53 mutant, proteinase3 (PR1), bcr-abl, tyrosinase, survivin, hTERT, sarcoma translocation breakpoints, EphA2, PAP, ML-IAP, AFP, EpCAM, ERG (TMPRSS2 ETS fusion gene), NA17, PAX3, ALK, androgen receptor, cyclin B 1, polysialic acid, MYCN, RhoC, TRP-2, GD3, fucosyl GMl, mesothelin, PSCA, MAGE Al, sLe(a), CYPIB I, PLACl, GM3, BORIS, Tn, GloboH, ETV6-AML, NY-BR-1, RGSS, SART3, STn, carbonic anhydrase IX, PAX5, OY-TES1, sperm protein 17, LCK, HMWMAA, AKAP-4, SSX2, XAGE 1, B7H3, legumain, Tie 2, VEGFR2, MAD-CT-1, FAP, PDGFR-β, MAD-CT-2, Notch1, ICAM1 and Fos-related antigen 1.

### Methods of Treatment

In certain embodiments, the methods embodied herein, identifying a mammal as having cancer. The methods include, whole genome sequencing of cfDNA fragments; mapping of the cfDNA fragments to a genomic origin and evaluating fragment length and obtaining genome-wide fragmentation profiles for each sample; identifying protein biomarkers of the subject; comparing the subject's cfDNA fragmentation profile and protein biomarkers with normal reference non-cancer subjects. The cfDNA fragmentation profile is compared to a reference cfDNA fragmentation profile, and identifying the mammal as having cancer when the cfDNA fragmentation profile in the sample obtained from the mammal is different from the reference cfDNA fragmentation profile.

In certain embodiments, a subject is diagnosed as having cancer, e.g. early stage cancer. In certain embodiments, the type of cancer is identified and the cancer is treated by various therapeutics, including therapeutics specific for the type of cancer. The cancer treatment can be surgery, adjuvant chemotherapy, neoadjuvant chemotherapy, radiation therapy, hormone therapy, cytotoxic therapy, immunotherapy, adoptive T cell therapy, targeted therapy, or any combinations thereof. The method also can include administering to the mammal a cancer treatment (e.g., surgery, adjuvant chemotherapy, neoadjuvant chemotherapy, radiation therapy, hormone therapy, cytotoxic therapy, immunotherapy, adoptive T cell therapy, targeted therapy, or any combinations thereof). The mammal can be monitored for the presence of cancer after administration of the cancer treatment.

Cancer therapies in general also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The chemotherapy may also be administered at low, continuous doses which is known as metronomic chemotherapy.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine;

acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells as well as genetically engineered variants of these cell types modified to express chimeric antigen receptors.

The immunotherapy may comprise suppression of T regulatory cells (Tregs), myeloid derived suppressor cells (MDSCs) and cancer associated fibroblasts (CAFs). In some embodiments, the immunotherapy is a tumor vaccine (e.g., whole tumor cell vaccines, peptides, and recombinant tumor associated antigen vaccines), or adoptive cellular therapies (ACT) (e.g., T cells, natural killer cells, TILs, and LAK cells). The T cells may be engineered with chimeric antigen receptors (CARs) or T cell receptors (TCRs) to specific tumor antigens. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. In some aspects, the T cells are activated CD4 and/or CD8 T cells in the individual which are characterized by γ-IFN-producing CD4 and/or CD8 T cells and/or enhanced cytolytic activity relative to prior to the administration of the combination. The CD4 and/or CD8 T cells may exhibit increased release of cytokines selected from the group consisting of IFN-γ, TNF-α and interleukins. The CD4 and/or CD8 T cells can be effector memory T cells. In certain embodiments, the CD4 and/or CD8 effector memory T cells are characterized by having the expression of CD44^{high} CD62L^{low}.

The immunotherapy may be a cancer vaccine comprising one or more cancer antigens, in particular a protein or an immunogenic fragment thereof, DNA or RNA encoding said cancer antigen, in particular a protein or an immunogenic fragment thereof, cancer cell lysates, and/or protein preparations from tumor cells. As used herein, a cancer antigen is an antigenic substance present in cancer cells. In principle, any protein produced in a cancer cell that has an abnormal structure due to mutation can act as a cancer antigen. In principle, cancer antigens can be products of mutated Oncogenes and tumor suppressor genes, products of other mutated genes, overexpressed or aberrantly expressed cellular proteins, cancer antigens produced by oncogenic viruses, oncofetal antigens, altered cell surface glycolipids and glycoproteins, or cell type-specific differentiation antigens. Examples of cancer antigens include the abnormal products of ras and p53 genes. Other examples include tissue differentiation antigens, mutant protein antigens, oncogenic viral antigens, cancer-testis antigens and vascular or stromal specific antigens. Tissue differentiation antigens are those that are specific to a certain type of tissue. Mutant protein antigens are likely to be much more specific to cancer cells because normal cells shouldn't contain these proteins. Normal cells will display the normal protein antigen on their MHC molecules, whereas cancer cells will display the mutant version. Some viral proteins are implicated in forming cancer, and some viral antigens are also cancer antigens. Cancer-testis antigens are antigens expressed primarily in the germ cells of the testes, but also in fetal ovaries and the trophoblast. Some cancer cells aberrantly express these proteins and therefore present these antigens, allowing attack by T-cells specific to these antigens. Exemplary antigens of this type are CTAG1 B and MAGEA1 as well as Rindopepimut, a 14-mer intradermal injectable peptide vaccine targeted against epidermal growth factor receptor (EGFR) v111 variant. Rindopepimut is particularly suitable for treating glioblastoma when used in combination with an inhibitor of the CD95/CD95L signaling system as described herein. Also, proteins that are normally produced in very low quantities, but whose production is dramatically increased in cancer cells, may trigger an immune response. An example of such a protein is the enzyme tyrosinase, which is required for melanin production. Normally tyrosinase is produced in minute quantities but its levels are very much elevated in melanoma cells. Oncofetal antigens are another important class of cancer antigens. Examples are alphafetoprotein (AFP) and carcinoembryonic antigen (CEA). These proteins are normally produced in the early stages of embryonic development and disappear by the time the immune system is fully developed. Thus self-tolerance does not develop against these antigens. Abnormal proteins are also produced by cells infected with oncoviruses, e.g. EBV and HPV. Cells infected by these viruses contain latent viral DNA which is transcribed and the resulting protein produces an immune response. A cancer vaccine may include a peptide cancer vaccine, which in some embodiments is a personalized peptide vaccine. In some embodiments. the peptide cancer vaccine is a multivalent long peptide vaccine, a multi-peptide vaccine, a peptide cocktail vaccine, a hybrid peptide vaccine, or a peptide-pulsed dendritic cell vaccine

The immunotherapy may be an antibody, such as part of a polyclonal antibody preparation, or may be a monoclonal antibody. The antibody may be a humanized antibody, a chimeric antibody, an antibody fragment, a bispecific antibody or a single chain antibody. An antibody as disclosed herein includes an antibody fragment, such as, but not limited to, Fab, Fab′ and F(ab′)2, Fd, single-chain Fvs (scFv), single-chain antibodies, disulfide-linked Fvs (sdfv) and fragments including either a VL or VH domain. In some aspects, the antibody or fragment thereof specifically binds epidermal growth factor receptor (EGFR1, Erb-B1), HER2/neu (Erb-B2), CD20, Vascular endothelial growth factor (VEGF), insulin-like growth factor receptor (IGF-1R), TRAIL-receptor, epithelial cell adhesion molecule, carcino-embryonic antigen, Prostate-specific membrane antigen, Mucin-1, CD30, CD33, or CD40.

Examples of monoclonal antibodies include, without limitation, trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-I131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™. (17-1A) (murine monoclonal antibody); Panorex (MAb17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Further examples of antibodies include Zanulimumab (anti-CD4 mAb), Keliximab (anti-CD4 mAb); Ipilimumab (MDX-101; anti-CTLA-4 mAb); Tremilimumab (anti-CTLA-4 mAb); (Daclizumab (anti-CD25/IL-2R mAb); Basiliximab (anti-CD25/IL-2R mAb); MDX-1106 (anti-PD1 mAb); antibody to GITR; GC1008 (anti-TGF-β antibody); metelimumab/CAT-192 (anti-TGF-β antibody); lerdelimumab/CAT-152 (anti-TGF-β antibody); ID11 (anti-TGF-β antibody); Denosumab (anti-RANKL mAb); BMS-663513 (humanized anti-4-1BB mAb); SGN-40 (humanized anti-CD40 mAb); CP870,893 (human anti-CD40 mAb); Infliximab (chimeric anti-TNF mAb; Adalimumab (human anti-TNF mAb); Certolizumab (humanized Fab anti-TNF); Golimumab (anti-TNF); Etanercept (Extracellular domain of TNFR fused to IgG1 Fc); Belatacept (Extracellular domain of CTLA-4 fused to Fc); Abatacept (Extracellular domain of CTLA-4 fused to Fc); Belimumab (anti-B Lymphocyte stimulator); Muromonab-CD3 (anti-CD3 mAb); Otelixizumab (anti-CD3 mAb); Teplizumab (anti-CD3 mAb); Tocilizumab (anti-IL6R mAb); REGN88 (anti-IL6R mAb); Ustekinumab (anti-IL-12/23 mAb); Briakinumab (anti-IL-12/23 mAb); Natalizumab (anti-α4 integrin); Vedolizumab (anti-α4 β7 integrin mAb); T1 h (anti-CD6 mAb); Epratuzumab (anti-CD22 mAb); Efalizumab (anti-CD11a mAb); and Atacicept (extracellular domain of transmembrane activator and calcium-modulating ligand interactor fused with Fc).

### EXAMPLES

### Example 1: Early Detection of Lung Cancer Using Cell-Free DNA Fragmentation

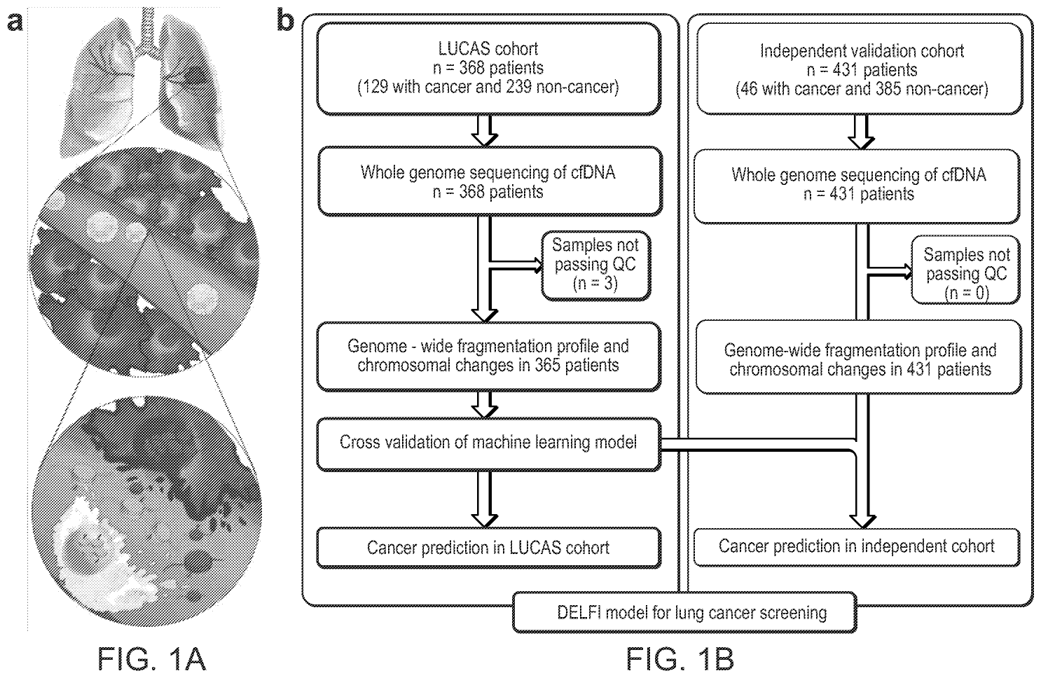

The rapid technological and analytical advancements in liquid biopsy analyses have identified cancer-related features in the cfDNA fragments in peripheral blood and have provided a new avenue for noninvasive detection of cancer. Mutations or methylation in circulating tumor DNA (ctDNA) can be directly detected in early stage lung cancer patients without prior knowledge of these alterations in tumors¹⁶⁻²¹. Given the relatively small number of sequence or epigenetic alterations that can be assessed by targeted high coverage sequencing, many individuals with cancer may be missed by such approaches and may also require sequencing of white blood cells (WBCs) to eliminate changes that result from clonal hematopoiesis^{17,18,22}. To increase the sensitivity of detection of early stage cancers a genome-wide approach was developed for analysis of cfDNA fragmentation profiles called DELFI (DNA evaluation of fragments for early interception)²³. This approach provides a view of the cfDNA “fragmentome”, permitting evaluation of the size distribution and frequency of millions of naturally occurring cfDNA fragments across the genome. As the cfDNA fragmentome can comprehensively represent both genomic and chromatin characteristics, it has the potential to identify a large number of tumor-derived changes in the circulation. In this study, this methodology was utilized for lung cancer detection and characterization in a prospectively collected real-world cohort comprising patients with malignant and benign pulmonary nodules as well as non-cancer individuals, including those with other clinical conditions (FIGS. 1A, 1B). Through this effort, a framework for incorporating noninvasive liquid biopsies in the clinic is provided, combining cfDNA fragmentation profiles with other markers and LDCT for lung cancer detection.

### Methods

Study Population Analyzed

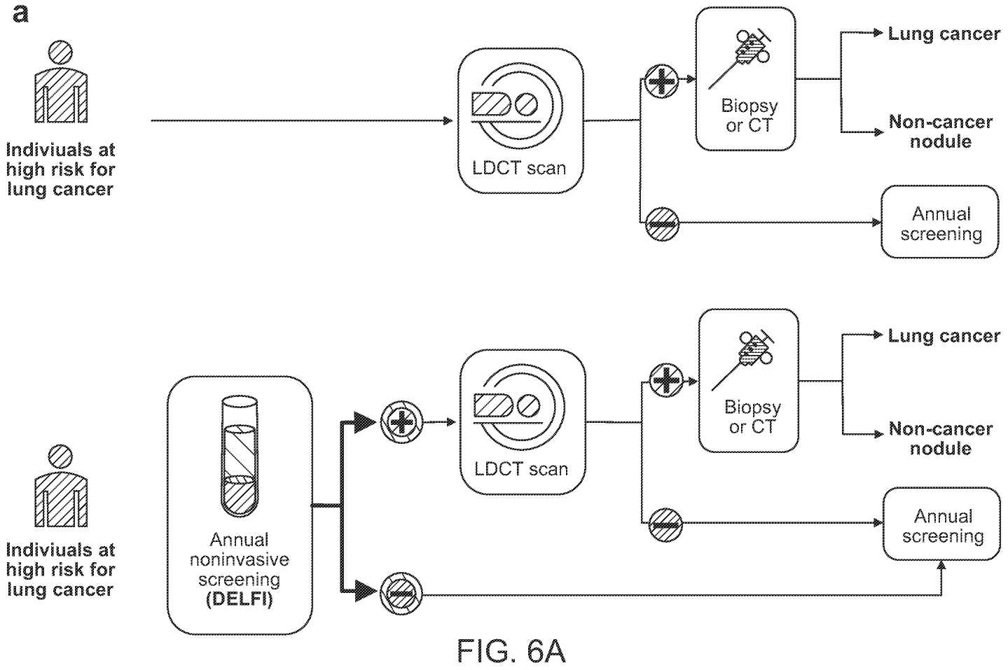

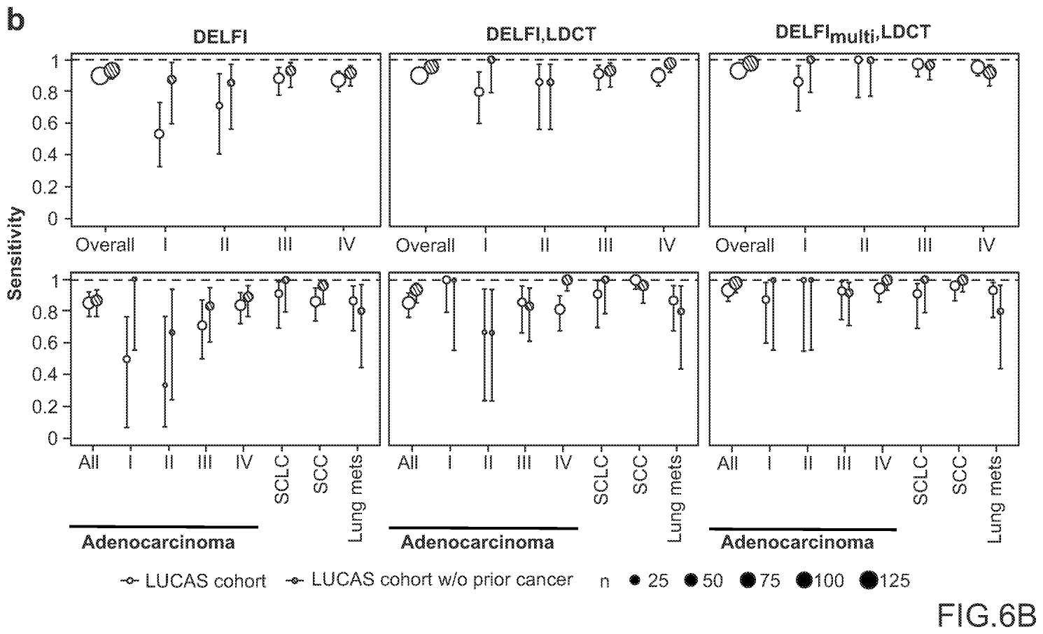

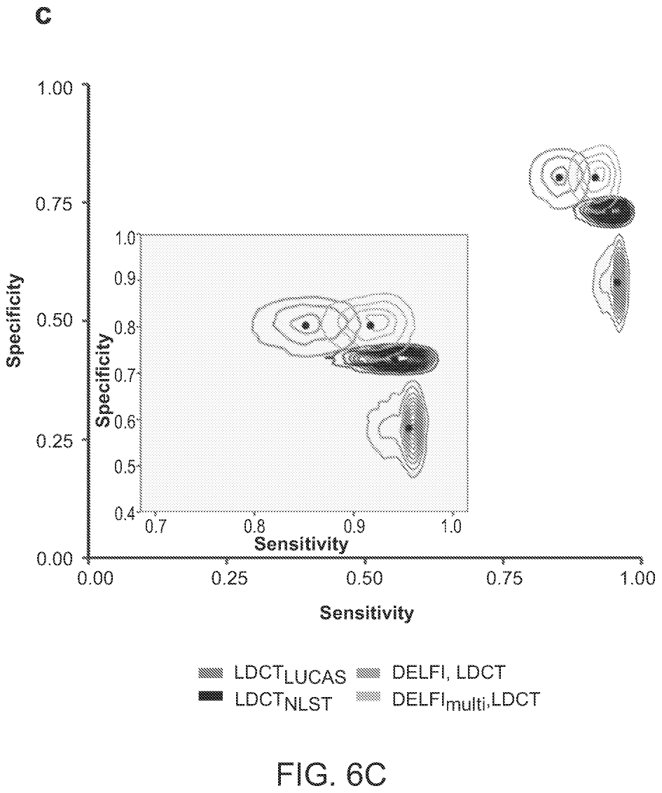

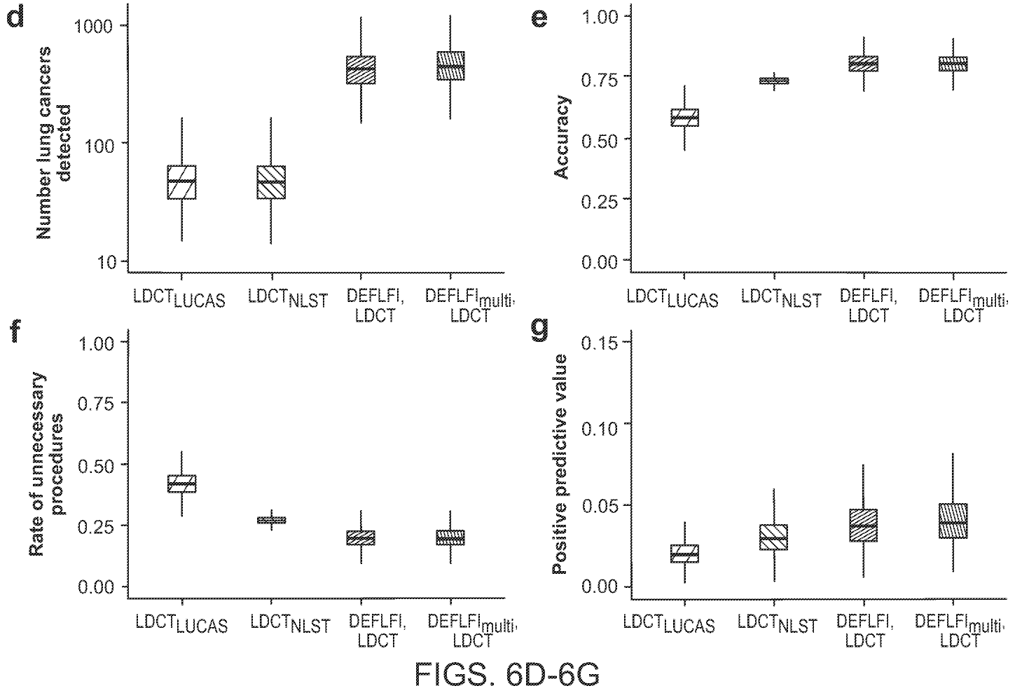

The LUCAS diagnostic cohort is a prospectively collected cohort of 368 predominantly symptomatic patients that presented in the Department of Respiratory Medicine, Infiltrate Unite, Bispebjerg Hospital, Copenhagen with a positive imaging finding on a chest X-ray or a chest CT. The study was conducted over 7 months from September 2012 to March 2013, and all patients had a clinical follow up until death or April, 2020. All patients had blood samples collected at their first clinic visit before the possible diagnosis of lung cancer was made. Samples from 365 patients that passed quality control from genomic sequencing were included in subsequent analyses. The analyzed cohort included 158 patients with no prior, baseline or future cancers, 129 patients with baseline lung cancer, and 78 patients without cancer at the time of blood collection, but with either earlier or later cancers (FIG. 6, Table 1). The validation cohort consisted of 385 non-cancer individuals from two screening cohorts for colorectal cancer in Denmark and the Netherlands and 46 patients with pathologically confirmed predominantly early stage lung cancer from an independent prospective collection through BioIVT (Westbury, NY). The validation cohort of lung cancer patients had a new diagnosis of lung cancer at the time of collection.

Sample Collection and Preservation

The sample collection for the LUCAS cohort was obtained at the time of the screening visit and performed as follows: venous peripheral blood was collected in one K2-EDTA tube and two serum gel tubes. Within two hours from blood collection tubes were centrifuged at 2330 g at 4° C. for 10 min After centrifugation, EDTA plasma and serum were aliquoted and stored at −80° C. for cfDNA and protein analyses, respectively.

For the validation cohort, venous peripheral blood for each individual was collected in one EDTA tube. Tubes were centrifuged at low speed (1500-3000 g) for 10-15 min within two hours from blood collection. The plasma portion from the first spin was spun a second time for 10 min. After centrifugation EDTA plasma was aliquoted and stored at −80° C. for cfDNA analyses.

Sequencing Library Preparation

Circulating cell-free DNA was isolated from 2-4 ml of plasma using the Qiagen QIAamp Circulating Nucleic Acids Kit (Qiagen GmbH), eluted in 52 μl of RNase-free water containing 0.04% sodium azide (Qiagen GmbH), and stored in LoBind tubes (Eppendorf AG) at −20° C. Concentration and quality of cfDNA were assessed using the Bioanalyzer 2100 (Agilent Technologies).

Next-generation sequencing (NGS) cfDNA libraries were prepared for WGS using 15 ng cfDNA when available, or entire purified amount when less than 15 ng. For the validation cohort available cfDNA up to 125 ng was used as input material for library preparation. In brief, genomic libraries were prepared using the NEBNext DNA Library Prep Kit for Illumina (New England Biolabs (NEB)) with four main modifications to the manufacturer's guidelines: (i) the library purification steps use the on-bead AMPure XP (Beckman Coulter) approach to minimize sample loss during elution and tube transfer steps; (ii) NEBNext End Repair, A-tailing and adaptor ligation enzyme and buffer volumes were adjusted as appropriate to accommodate the on-bead AMPure XP purification strategy; (iii) Illumina dual index adaptors were used in the ligation reaction; and (iv) cfDNA libraries were amplified with Phusion Hot Start Polymerase. All samples underwent a 4 cycle PCR amplification after the DNA ligation step.

In total, 23 batches of cfDNA library preparations were performed for the LUCAS cohort. Each batch included a combination of cancer patients and non-cancer controls. All batches included a technical replicate of nucleosomal DNA obtained from nuclease-digested human peripheral blood monocytes (PBMCs) to assess sequencing consistency across batches performed on a different date. A negative library control was periodically included where buffer TE pH 8.0 was used instead of a DNA sample to assure there was no DNA contamination during the library preparation. The validation cohort was prepared in the same fashion as above and the 485 samples, including embodiments and controls, were spread over 33 batches.

Low Coverage Whole Genome Sequencing and Alignment

Whole-genome libraries of cancer patients and cancer-free individuals were sequenced using 100-bp paired-end runs (200 cycles) on the Illumina HiSeq2500 platform at 1-2× coverage per genome. Prior to alignment, adapter sequences were filtered from reads using the fastp software³⁴. Sequence reads were aligned against the hg19 human reference genome using Bowtie2³⁵ and duplicate reads were removed using Sambamba³⁶. Post-alignment, each aligned pair was converted to a genomic interval representing the sequenced DNA fragment using bedtools³⁷. Only reads with a mapq score of at least 30 or greater were retained. Read pairs were further filtered if overlapping a problematic region provided by the Duke Excluded Regions blacklist (genome.ucsc.edu/cgi-bin/hgTrackUi?db=hg19&g=wgEncodeMapability; Amemiya, H. M., Kundaje, A. & Boyle, A. P. The ENCODE Blacklist: Identification of Problematic Regions of the Genome. Sci Rep 9, 9354 (2019). https://doi.org/10.1038/s41598-019-45839-z). To capture large-scale epigenetic differences in fragmentation across the genome estimable from low-coverage whole genome sequencing, the hg19 reference genome was tiled into 473 non-overlapping 5 Mb bins spanning approximately 2.4 GB of the genome. All bins had an average GC content >=0.3 and an average mappability >=0.9.

Whole Genome Fragment Features

Ratios of the number of short (100 to 150 bp) to long (151 to 220 bp) fragments across the 473 bins were normalized for GC-content and library size. As GC-related biases have been largely attributed to preferential amplification of fragments during PCR⁴⁰, a non-parametric method was developed for fragment-level GC adjustment. For each individual in the LUCAS cohort, fragments were assigned to one of 100 possible GC strata between 0 and 1 (1 indicating a fragment with all G and C nucleotides), obtaining the total number of fragments within each GC stratum. In the same manner, a distribution of fragment counts was obtained by GC stratum for the held-out set of 54 non-cancer samples as well as the median of the 54 distributions that were referred to as a target distribution. To normalize sample-to-sample PCR biases in LUCAS, the collection of fragments in GC stratum i were assigned a weight wᵢ such that Σᵢ₌₁^{N}^{ᵢ} wᵢ=tᵢ where tᵢ denotes the number of fragments in the target distribution, N_i the total number of fragments in stratum i, and i=1, . . . 100. The GC-adjusted number of short and long fragments were computed for each 5 Mb bin as the sum of the weights for the fragments aligned to that bin, thereby normalizing both sample-to-sample variation in GC-biases as well as differences in library size. Fragmentation profiles of the GC-adjusted short to long ratios were standardized to have mean zero and unit standard deviation across the genome.

In addition to fragmentation profiles, z-scores were also computed for chromosomal arms and a genome-wide summary of the overall cfDNA fragmentation. Z-scores for each of the 39 autosomal arms were obtained by centering and scaling the total GC-adjusted fragment count for each arm by the mean and standard deviation of the corresponding arm-specific counts in the 54 non-cancer samples used as a reference set^{23,41}. To summarize the overall cfDNA fragmentation sizes for each sample, we computed the ratio of multinucleosomal (>=250 bp) to mononucleosomal (<250 bp) fragments.

Analysis Based on Public Databases from TCGA

Copy number data from the two lung cancer cohorts in TCGA (LUAD n=518 and LUSC n=501) were retrieved using the package RTCGA v1.16.0⁵¹. The tumor to normal log copy ratio values were compared to tumor type specific thresholds⁵² to identify genomic regions harboring copy gains and losses. The copy number status in each of the 5 Mb bins across the genome was determined by requiring a minimum coverage of 90% of the bin interval by segments harboring a gain or loss. The frequency of copy gain and loss in the genomic bins were calculated for each of the lung cancer cohorts in TCGA.

Machine Learning and Cross-Validation Analyses

Five-fold cross-validation was used to develop a predictive model for early and late stage cancer detection where feature selection and model development were evaluated on four of the five folds (training set) and a fifth held-out fold was used only to assess model performance (test set). The total number of samples available for training and testing include 158 participants with no prior, baseline or future cancer and 129 patients with cancer at the time of the blood draw. Patients with a prior or future cancer (n=78) were not used for training or testing. Due to the high dimensionality of the fragmentation features relative to the number of available samples for training, a principal components analysis was performed within each training set to reduce the dimensionality of the feature space, retaining the minimum number of principal components needed to explain 90% of the variance of the fragmentation profiles between samples. In addition to the principal component features, all 39 z-scores were evaluated in a logistic regression model with a LASSO penalty. The optimized LASSO penalty of 0.0017 in the analysis was obtained by resampling using the caret R package. The DELFI score derived for each sample corresponds to the mean score across the 10 cross validation repeats. As both the feature selection (principal components analysis) and LASSO model were evaluated only on the samples in the training set, this approach provides an unbiased estimate of model performance.

As an additional measure of model performance, a final model was obtained using all 158 non-cancer individuals and 129 patients with baseline lung cancer in the LUCAS study and evaluated this fixed model in an independent cohort (validation set) of non-cancer individuals (n=385) and predominantly early stage cancer (n=46). Using the fixed model, a prediction score was obtained for each individual in the validation set and classified each individual as non-cancer or cancer using a fixed cutoff from LUCAS that provided a desired specificity of 80%. Notably, the samples from the validation cohort were entirely batch-independent from the LUCAS cohort with respect to sample collection, library preparation, and sequencing.

To assess whether clinical and serum protein markers in addition to fragmentation features could further improve prediction, a multimodal predictive model was evaluated using the repeated five-fold cross-validation approach. Fragmentation features summarized by a principal component analysis and z-scores were evaluated as described above such that both feature selection and estimation of model parameters were independent of the test set. For clinical and serum protein markers, we included age, smoking history, COPD status, and CEA. A logistic regression model with a LASSO penalty was used to evaluate the fragmentation, clinical, and protein biomarker features in each training fold.

Treatment in the LUCAS Cohort

All patients were evaluated after diagnosis for eligibility for either 1) primary surgery, 2) concomitant chemotherapy and radiotherapy with curative intent, 3) standard palliative systemic oncological treatment (with either chemotherapy or targeted therapy), or 4) best supportive care—all according to the Danish national treatment guidelines for lung cancer in 2012-13, which were in concordance with the ESMO guidelines⁴²⁻⁴⁴.

All patients were evaluated for possible primary surgery based on TNM-stage as well as possible co-morbidities that might prevent the possibility for anesthesia. Patients underwent primary lung surgery for a solitary lung metastasis (two colo-rectal, one testis cancer, and one breast cancer), and a subset received post-surgery adjuvant chemotherapy according to ESMO guidelines in 2012-2013. If patients were not eligible for primary surgery, they were then evaluated at a multi-disciplinary team conference for concomitant chemotherapy (platinum dublet combined with either Vinorelbine (for NSCLC) or Etoposide (for SCLC)) and radiotherapy (either 2 Gray in 33 fractions, 5 F/W or stereotactic radiotherapy 15 Gray in 3 fractions (for NSCLC) or 2.05 Gray in 45 fractions or 3 Gray in 10 fractions (for SCLC)) with curative intent. Patients with poor ECOG performance status and/or significant co-morbidities were precluded from having any type of oncological treatment and were referred for supportive care. Patients with advanced disease at the time of diagnosis were eligible for palliative chemotherapy and/or radiotherapy. Patients with an EGFR mutation were primarily treated with Gefitinib in 1^{st} line and Erlotinib following either Gefitinib or chemotherapy and those with ALK-translocation were treated with crizotinib. Patients from the initial palliative treatment cohort went on to receive 2^{nd} line oncological treatment after progression of disease (typically Pemetrexed monotherapy). Only 16 patients received additional therapy after a 2^{nd} line treatment, with one patient receiving a total of seven lines of treatment. Since the cohort is from 2012-2013 only two patients received immunotherapy (Nivolumab) in respectively 3^{rd} and 7^{th} line in a CheckMate protocol.

Association of Clinical Covariates and Survival with the DELFI Score

Univariate analyses comparing the distribution of the DELFI score to baseline clinical and laboratory covariates age, smoking, and serum inflammatory markers was performed using a Wilcoxon rank sum test. Additionally, the relationship of the DELFI score and cancer risk with and without baseline covariates age, smoking, and sex was evaluated using logistic regression.

To assess whether the DELFI score was associated with prognosis, high risk lung cancer patients were categorized according to whether they were more likely to have cancer than not (DELFI score >0.5). To assess whether this categorization was associated with survival among lung cancer patients in a univariate analysis, a log rank test was used to compare survival curves. In addition to the univariate analysis, it was evaluated whether the DELFI score was independently associated with lung cancer specific survival in a multivariable Cox proportional hazards model that included histological subtypes of primary lung cancer and clinical staging as additional explanatory variables.

Genome-Wide Transcription Factor Analyses for Prediction of Histological Subtype

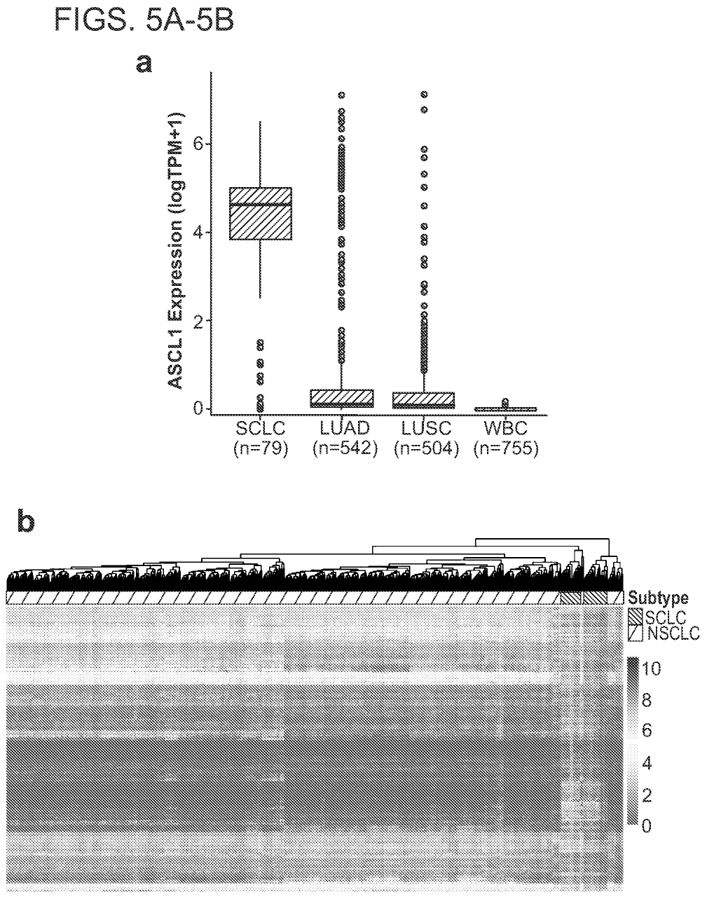

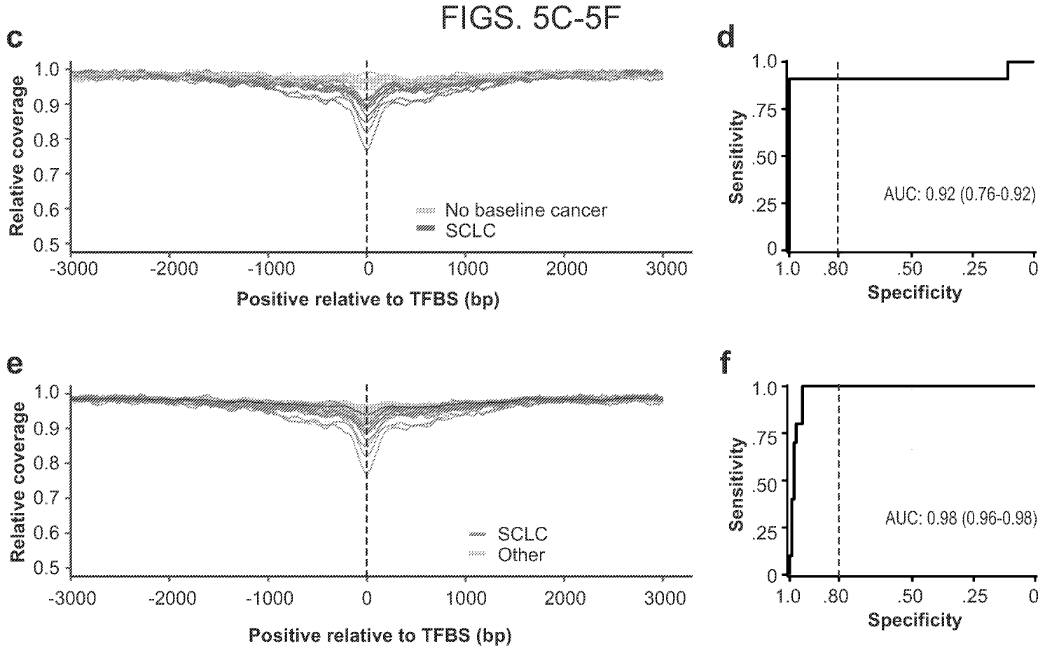

Gene expression values were obtained as raw counts using recount3 1.0.2⁵⁶ and converted to transcripts per million (TPM) using recount 1.16.1 for SCLC (n=79)⁵⁷, lung adenocarcinoma (n=542) and lung squamous cell carcinoma (n=504) generated by The Cancer Genome Atlas (TCGA), and whole blood (n=755) generated by the Genotype-Tissue Expression (GTEx) project. The median TPM value was computed for 1,639 transcription factors (TFs)⁵⁸ in each cancer/tissue type. TFs that were unexpressed (median TPM <1) were identified in lung adenocarcinoma, lung squamous cell carcinoma, and whole blood, and then ordered them from highest to lowest expression in SCLC. The top gene was ASCL1 (median TPM=101). Chromatin immunoprecipitation was then obtained followed by sequencing (ChIP-seq) peaks for ASCL1 (n=13,920 peaks) (GEO Sample accession number: GSM3704421)⁵⁹. For each peak in in the autosomes (n=13,693 peaks) the center of the peak was defined as position 0 and then the coverage was computed in a +/−3,000 bp window around each peak separately for 125 samples with a DELFI score of at least 0.37 (corresponding to a specificity of 85%). A small number of peaks were excluded with an average coverage of >3 across samples. The mean of the coverages at each position (−3,000 to +3,000) across all peaks was computed for each sample. For FIGS. 5C, 5E the relative coverage for a given sample was computed by taking the coverage at each position in the +/−3,000 bp window surrounding the ASCL1 binding sites and dividing by the maximum within that sample. The SCLC samples are plotted separately, and the samples in the ‘Other’ group are plotted as the median relative coverage or fragment length (black line) and the 0.05 and 0.95 quantiles of the relative coverage or fragment length at each position relative to the ASCL1 binding sites (shaded region). For the ROC curves (FIGS. 5D, 5F), relative coverage was computed for each sample as the mean coverage in a +/−100 bp window surrounding the center of the ASCL1 binding sites divided by the mean coverage in a +/−250 bp window surrounding 2,750 bp upstream and downstream of the binding sites. The ROC curve was generated using pROC 1.16.2⁶⁰. For FIG. 5B, names of 620 ASCL1 target genes was obtained⁶¹, 600 of which had a matching gene name in the gene expression datasets. For these 600 genes, a gene was defined as ‘Overexpressed’ in a given sample when TPM value >3 standard deviations above the mean for that gene across all samples.

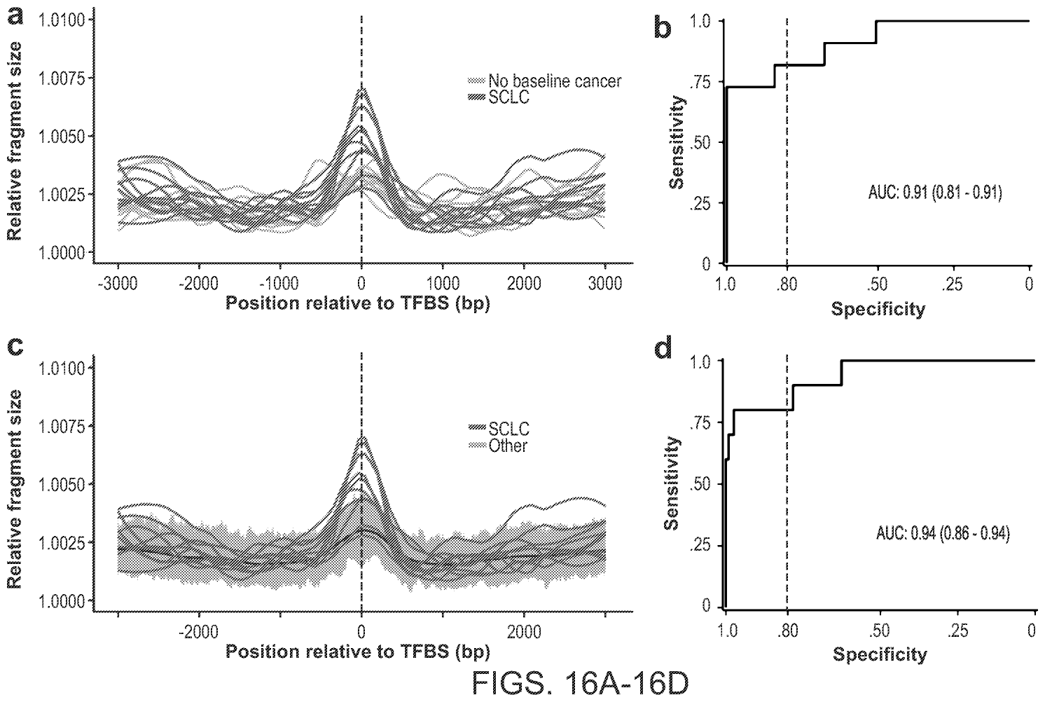

Using fragments <200 bp, the mean fragment size was computed at each position in a +/−3,000 bp window surrounding the ASCL1 binding sites. For FIG. 16A, the SCLC samples and 10 random no baseline cancer samples are plotted separately and smoothed using the LOESS method. For FIG. 16C the relative fragment size for a given sample is computed by taking the average fragment size at each position in the +/−3,000 bp window surrounding the ASCL1 binding sites and dividing it by the minimum within that sample. For FIG. 16D, the SCLC samples are plotted separately and smoothed using the LOESS method, and the samples in the ‘Other’ group are plotted as the median relative fragment size (black line) and the 0.05 and 0.95 quantiles of the relative fragment size at each position relative to the ASCL1 binding sites (shaded region). For ROC curves, relative fragment length was computed for each sample as the mean fragment size in a +/−100 bp window surrounding the center of the ASCL1 binding sites divided by the mean fragment size in a +/−250 bp window surrounding 1,250 bp upstream and downstream of the binding sites.

Modelling of DELFI Performance in a Screening Population

To normalize sample-to-sample PCR biases in LUCAS, the collection of fragments in GC stratum i were assigned a weight wᵢ such that Σᵢ₌₁^{N}^{ᵢ} wᵢ=tᵢ where tᵢ denotes the number of fragments in the target distribution and Nᵢ the total number of fragments in GC stratum i.

To assess performance of LDCT alone and DELFI followed by LDCT in a hypothetical screening population of 100,000 individuals, we used Monte Carlo simulations to capture uncertainty of unknown parameters sensitivity, specificity, adherence, and lung cancer prevalence. Prior models of sensitivity for LUCAS alone were centered loosely on empirical estimates from the LUCAS and NLST cohorts:

$${θ}_{1 , M} \sim \{ \begin{matrix}π \times N ( 0.96 , 0.005 ) + ( 1 - π ) \times n ( 0.94 , 0.02 ) & M = LD C {T}_{LUCAS} \\ Beta ( 93. 8 , 6 \text{.2} ) & M = LD C {T}_{N L S T} \\ Beta ( 85 , 15 ) & M = DE L FI , LDCT \\ Beta ( 91 , 9 ) & M = DE L F {I}_{multi} , LDCT\end{matrix} .$$

We sampled π˜Bernoulli(0.5).
For specificity, prior models were

$${θ}_{2 , M} ∼ \{ \begin{matrix}Beta ( 58 , 42 ) & M = LDC {T}_{LUCAS} \\ Beta ( 93.8 , 6.2 ) & M = LDC {T}_{N L S T} \\ Beta ( 86 , 14 ) & M = DEL FI , LDCT \\ Beta ( 94 , 6 ) & M = DEL F {I}_{multi} , L D C T\end{matrix}$$

The number of individuals screened in our simulated screening study depends on adherence to screening guidelines. Letting n denote the size of our screening study, our sampling model for n is given by

n˜Binomial(10⁵,η)

η˜beta(aₙ,βₙ)

For LDCT alone, shape parameters αₙ and βₙ were 12 and 188⁷ while for DELFIₘᵤₗₜᵢ followed by LDCT shape parameters were 80 and 20³². Conditional on the size of our screening study and draws of θ_{1,M} and θ_{2,M} from their respective prior distributions, we sampled the disease status, y, and screening results, x, conditional on y:

yᵢ˜Bernoulli(ψ) for i=1, . . . ,n

ψ˜Beta(9.1,990.9)

xᵢ|{yᵢ=1,M}˜Bernoulli(θ_{1,M})

xᵢ|{yᵢ=0,M}—Bernoulli(1−θ_{2,M})

The informative prior for prevalence, ψ, in our hypothetical population ensures that our screening study will be comprised predominantly of individuals without cancer, but allows the true prevalence to be smaller or larger that the estimate of 0.91% from the NLST study⁵. The number of patients with lung cancers detected, accuracy, false positive rate, and positive predictive value were calculated from the joint distribution of x and y. We repeated the above sampling procedure 10,000 times, thereby obtaining predictive distributions for these statistics that reflect uncertainty of sensitivity, specificity, adherence, and prevalence.

Bioinformatic and Statistical Software

All statistical analyses were performed using R version 3.6.1. After trimming of adapter sequences using fastp (0.20.0), we used Bowtie2 (2.3.0) to align paired end reads to the hg19 reference genome. PCR duplicates were removed using Sambamba (0.6.8) and the remaining aligned read pairs were converted to a bed format using Bedtools (2.29.0). The R package data.table (1.12.8) was used for manipulation of tabular data and binning fragments in 5 Mb windows along the genome. The R packages caret (6.0.84) and gbm (2.1.5) were used to implement the classification by gradient boosted trees and resampling.

### Results

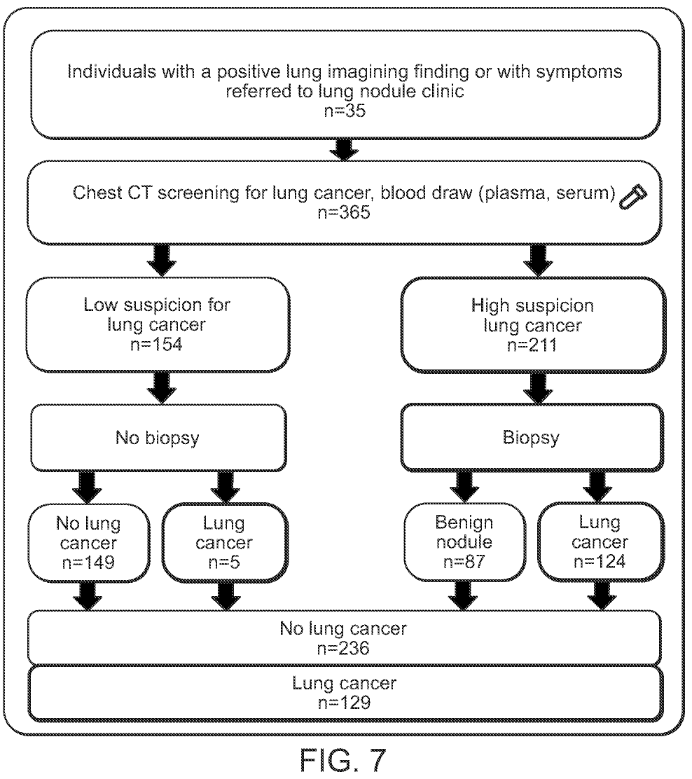

Patient blood samples from a prospective diagnostic study of 365 individuals conducted at Bispebjerg Hospital in Copenhagen, Denmark (LUCAS cohort), were examined. during a seven month period. The majority of subjects in the cohort were symptomatic individuals at high-risk for lung cancer (age 50-80 and smoking history >20 pack-years) (Table 1). The cohort included 323 subjects (90%) with pulmonary, non-pulmonary or constitutional symptoms, with the majority having common smoking-related symptoms such as cough or dyspnea. The remainder were asymptomatic at enrollment with an incidental chest image finding by X-ray or CT that was suspicious for lung malignancy. At the time of the patient's clinic visit an additional chest CT or 18F-PET/CT was performed to assess the identified nodule or infiltrate (FIG. 7). Of the 365 individuals studied, 129 were determined to have lung cancer a few days after the time of the blood collection (median 9.5 days, range 0-44) while the remainder had histologically proven benign nodules (n=87) or were not biopsied due to low clinical and radiographic suspicion for cancer (n=149)(FIG. 7). Standard algorithms for management of pulmonary nodules, including the Fleischner Society pulmonary nodule recommendations²⁴⁻²⁶, were used to determine clinical management.

DELFI Performance for Lung Cancer Detection

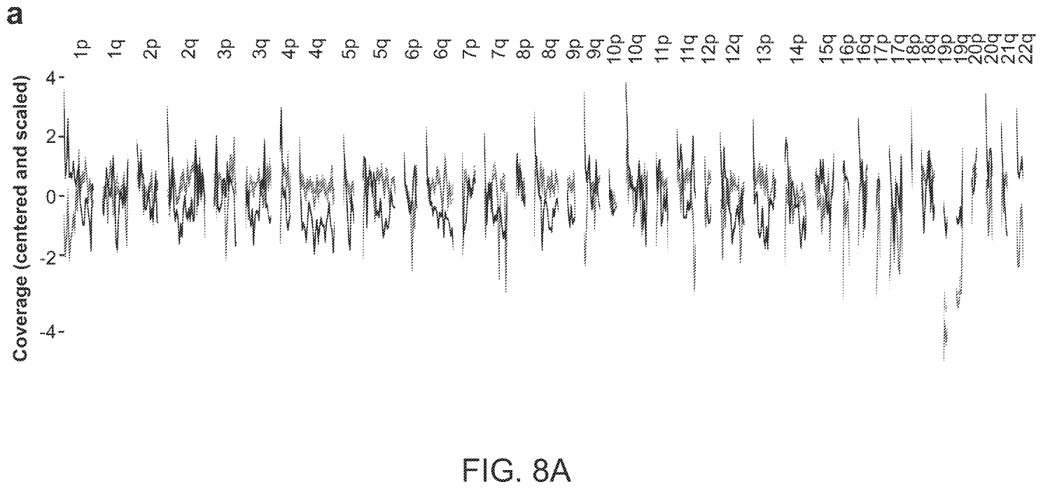

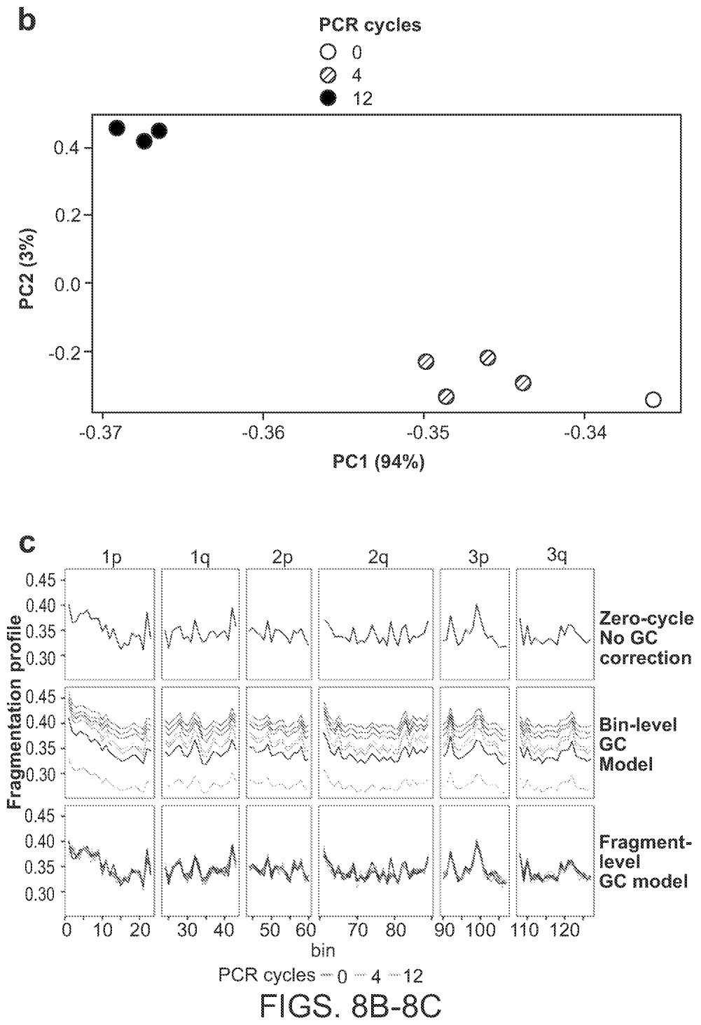

2-4 ml of plasma was isolated from each patient in the LUCAS cohort and the extracted cfDNA was examined using the DELFI approach with experimental and bioinformatic improvements. As PCR is known to affect the representation of amplified genomic fragments depending on GC content and fragment length, DELFI genome-wide fragmentation profiles were evaluated using genomic libraries created without amplification or with 4 or 12 cycles of PCR. It was found that libraries created with 4 cycles of PCR had profiles that were similar to those without any amplification, while 12 cycles led to substantial biases (FIGS. 8A, 8B). A novel fragment-based GC correction method was developed that simultaneously accounts for preferential amplification by fragment length and/or GC content (see Methods). It was examined whether this approach among 4 cycle libraries would further minimize GC biases compared to a commonly used bin-based approach²⁷ and found that the fragment-based approach was closest to the libraries without amplification (FIG. 8C). A 4 cycle amplification was therefore used to generate genomic libraries, and sequenced the cfDNA fragments using shallow whole genome sequencing (˜2× coverage) with an average of 40 million paired reads per sample (FIG. 1). To examine genome-wide cfDNA fragmentation patterns, the fragment-based GC corrected sequence data was used to evaluate fragmentation profiles across the genome in 473 non-overlapping 5 MB regions with high mappability, each region comprising ˜80,000 fragments, and spanning approximately 2.4 GB of the genome.

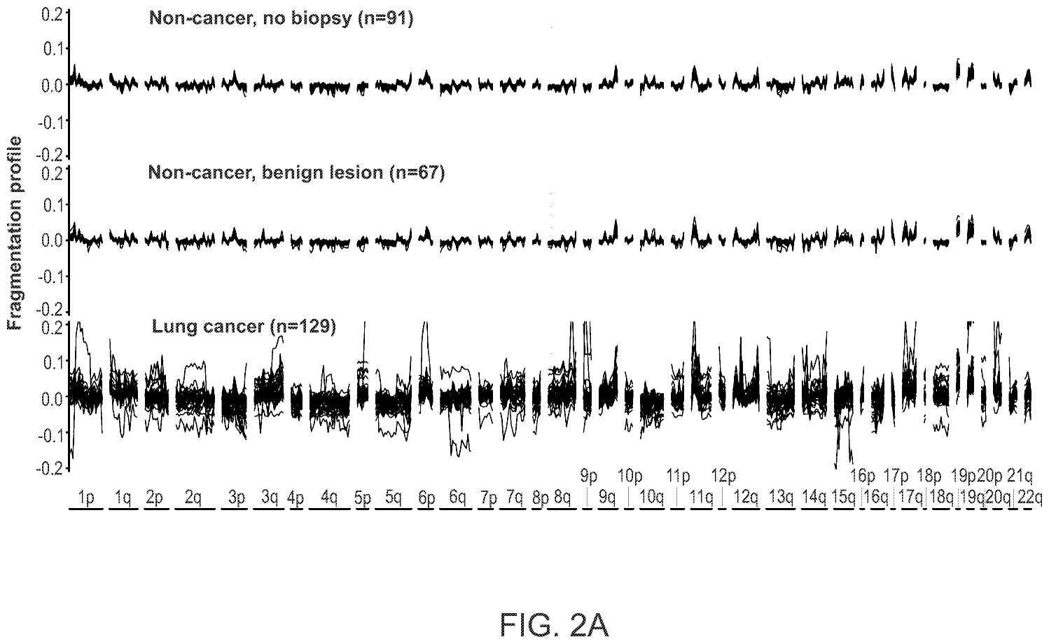

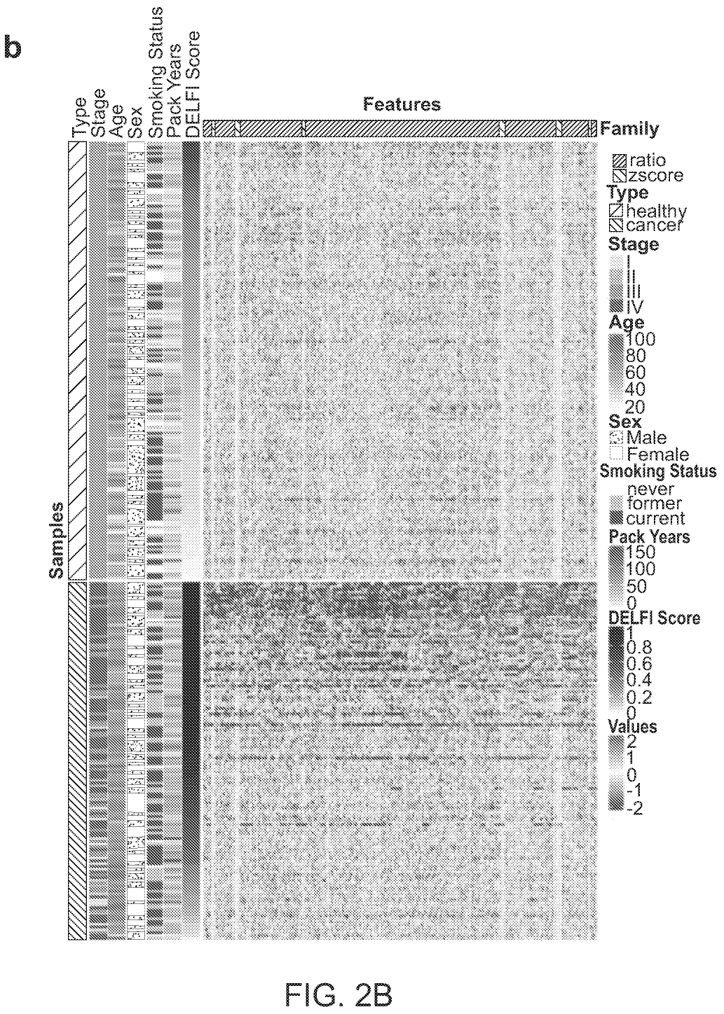

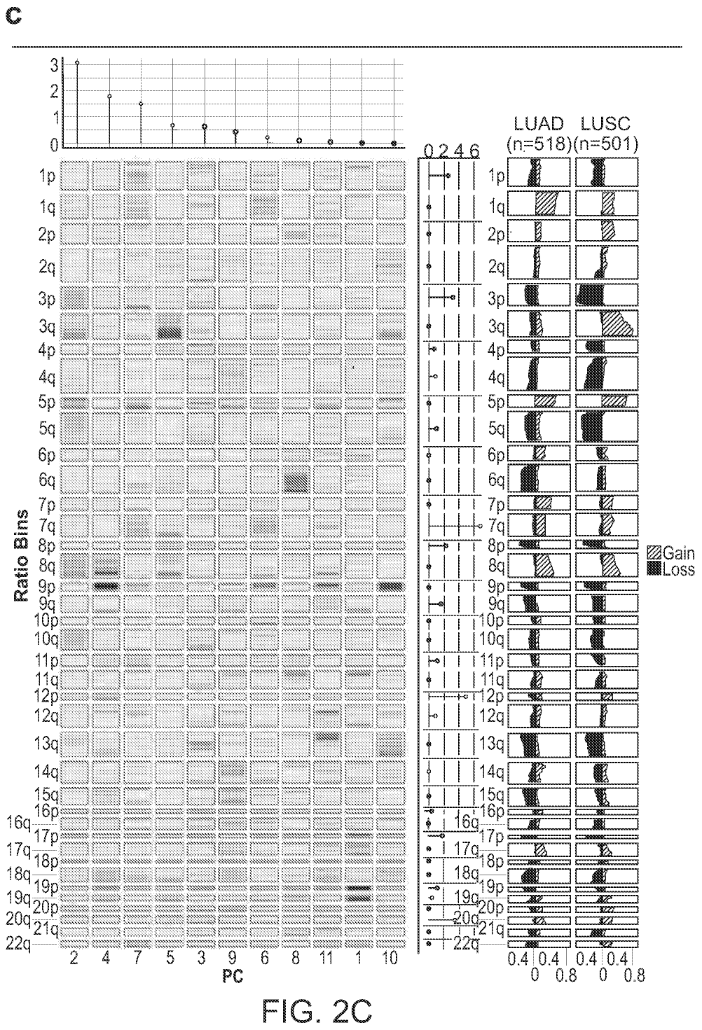

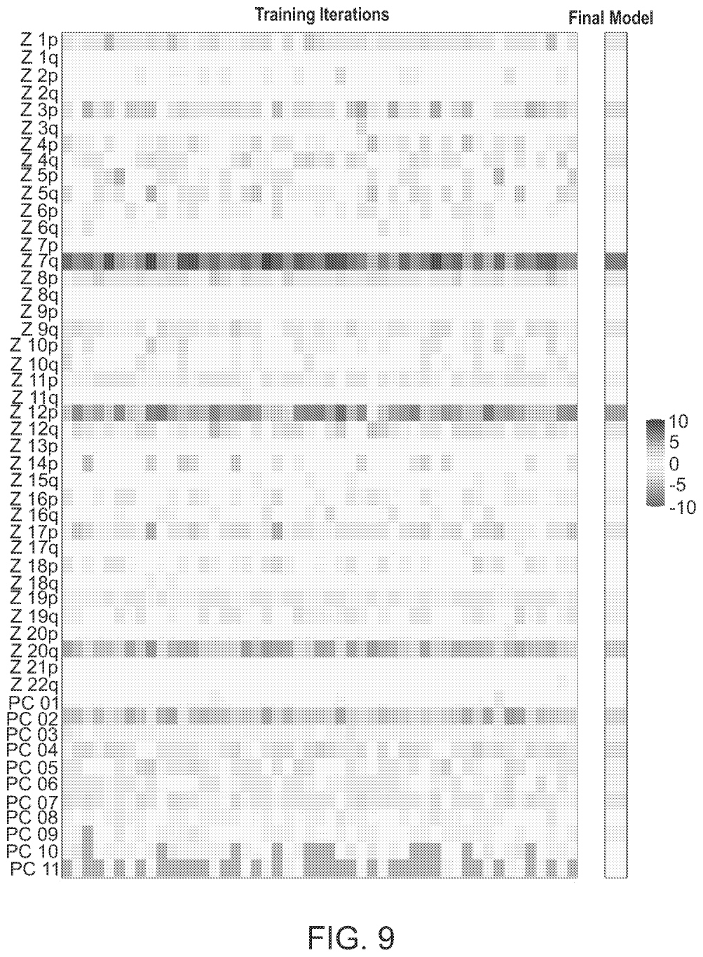

The resulting fragmentation profiles were remarkably consistent among non-cancer individuals, including those with non-malignant lung nodules (FIGS. 2A, 2B). In contrast, cancer patients displayed widespread genome-wide variation (FIGS. 2A, 2B). Remarkably, the fragmentation profile differences could be observed in multiple regions throughout the genome for the majority of cancer patients, including across stages and histologies. A machine learning model was employed to examine whether cfDNA profiles had characteristics of an individual with or without lung cancer. Due to the high dimensionality of our genome-wide fragmentation profiles relative to the number of patients analyzed, a principal component analysis (PCA) was performed to identify linear combinations of our fragmentation features that explained at least 90% of the variance. This dimensionality reduction step was incorporated into a machine learning model and the performance characteristics were estimated by repeated fivefold cross-validation, generating a score for each individual as an average over the cross-validation repeats (DELFI score). Analysis of the features incorporated in the machine learning models and corresponding measures of variable importance revealed fragmentation and chromosomal changes that were altered in cancer patients and predictive of cancer risk (FIG. 2C). The importance of these features were consistent across the training folds (FIG. 9). Among the genomic changes incorporated in the model, chromosomal arms that were increased or decreased in cfDNA representation corresponded to those commonly gained or lost in lung cancer as seen in previous TCGA large-scale genomic studies for lung adenocarcinoma (n=518) and squamous cell carcinoma (n=501) (FIG. 2C). These included increased cfDNA levels of 7q, 12p, and 20q, or decreased levels of 1p, 3p, 8p and 17p, all known to be gained or lost, respectively, in a variety of lung cancers²⁸⁻³⁰.

Combining DELFI Profiles with Multimodal Analyses

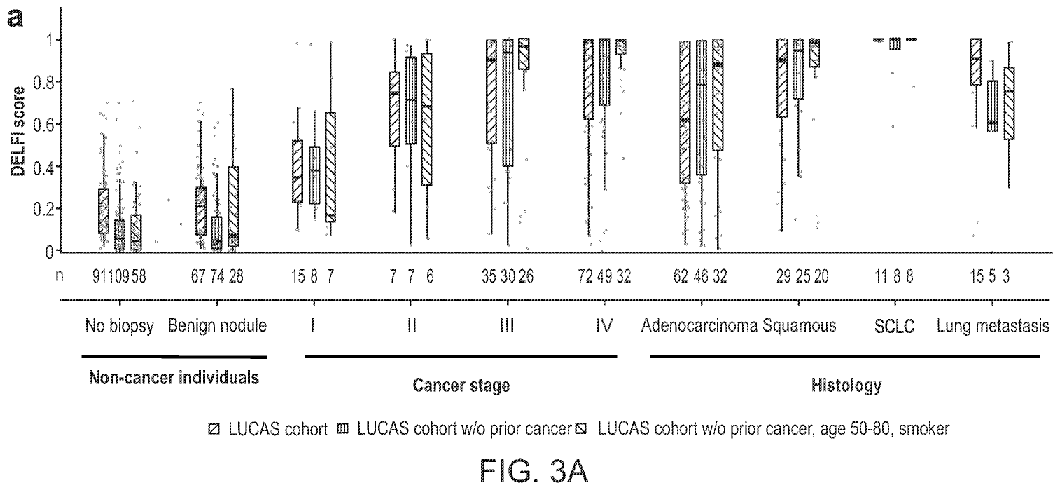

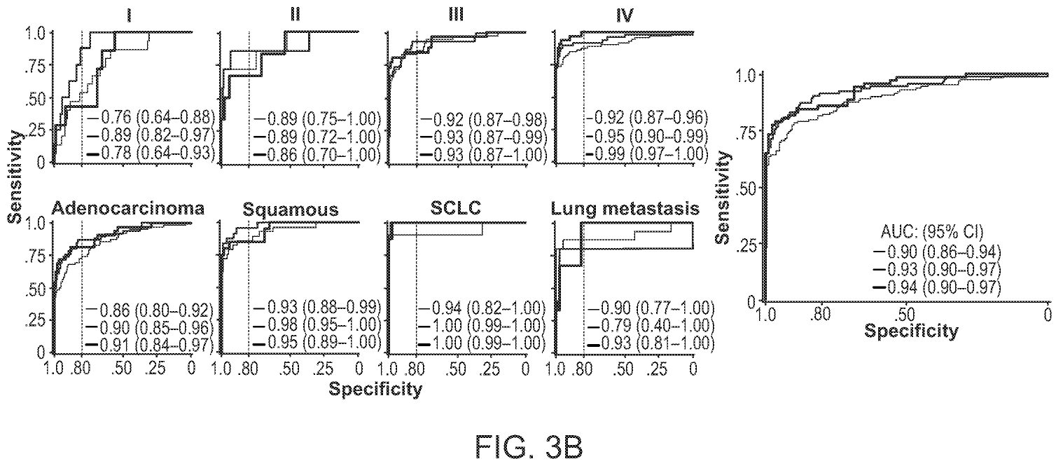

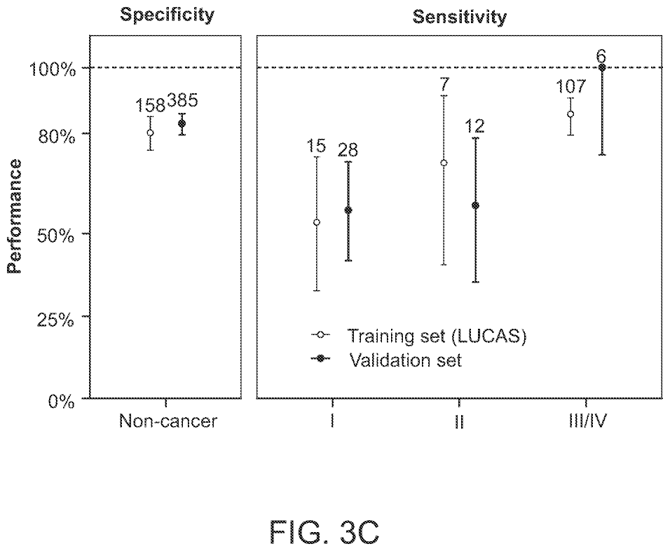

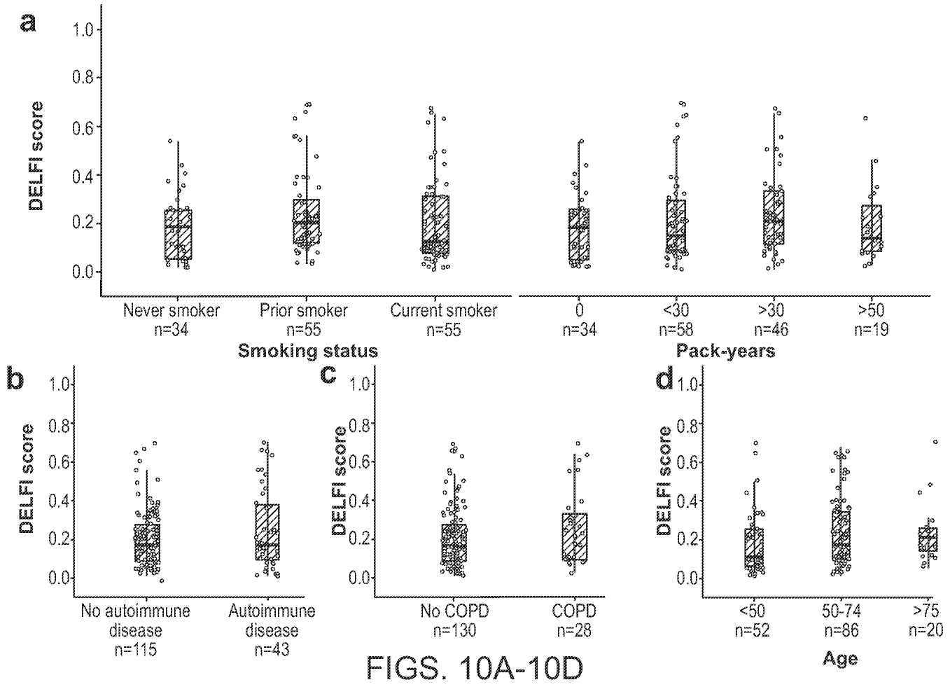

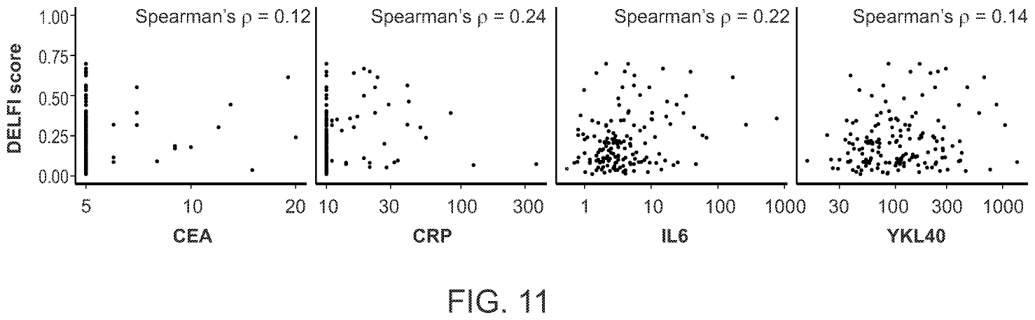

As clinical characteristics may affect tumor biomarkers, it was first sought to investigate whether non-malignant nodules, demographic parameters such as age or smoking history, or the presence of chronic obstructive pulmonary disease (COPD) or autoimmune diseases were associated with DELFI scores. An unbiased analysis of these characteristics was possible because of the prospective observational trial collection of the LUCAS cohort. No difference in the DELFI score was observed when comparing non-cancer individuals with or without benign lung lesions (median DELFI score 0.16 vs 0.21, p=0.99, Wilcoxon rank sum test, FIG. 3A). Changes in the DELFI score were also not observed among age groups (F statistic=1.65, p=0.20), among current, prior, and never-smokers (F statistic=1.3, p=0.27), and across pack-years in non-cancer individuals (F statistic=0.67, p=0.57) (FIGS. 10A-10D). Similarly, differences were not observed between patients with or without COPD (p=0.26) or patients with or without autoimmune diseases (p=0.38). Finally, a correlation was not observed between the levels of the inflammatory markers CRP or IL-6 and DELFI score in cancer-free individuals, consistent with the notion that altered cfDNA fragmentation is unique to cancer and the DELFI score is not affected by the presence of acute or chronic inflammatory conditions (FIG. 11).

DELFI Analyses of Lung Cancer Progression and Outcome

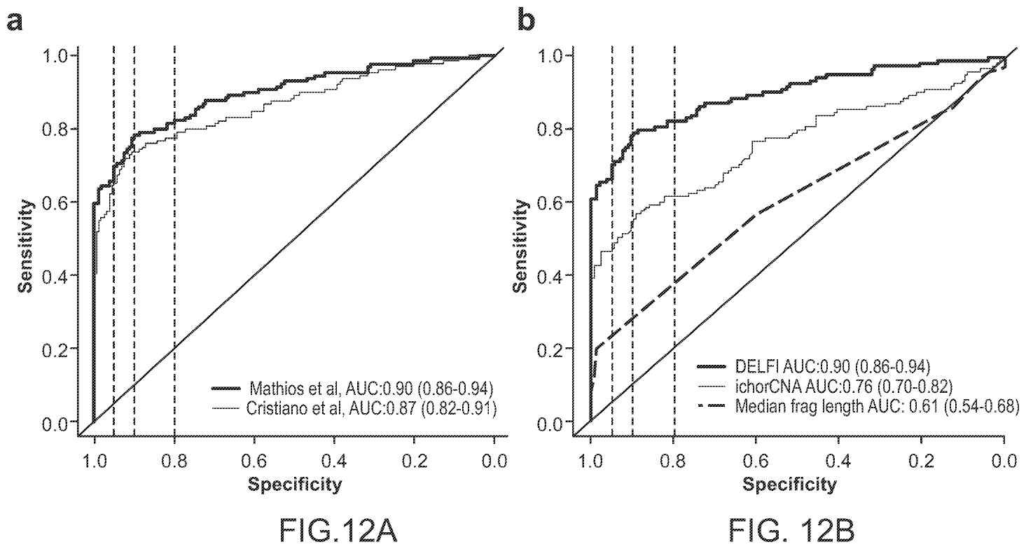

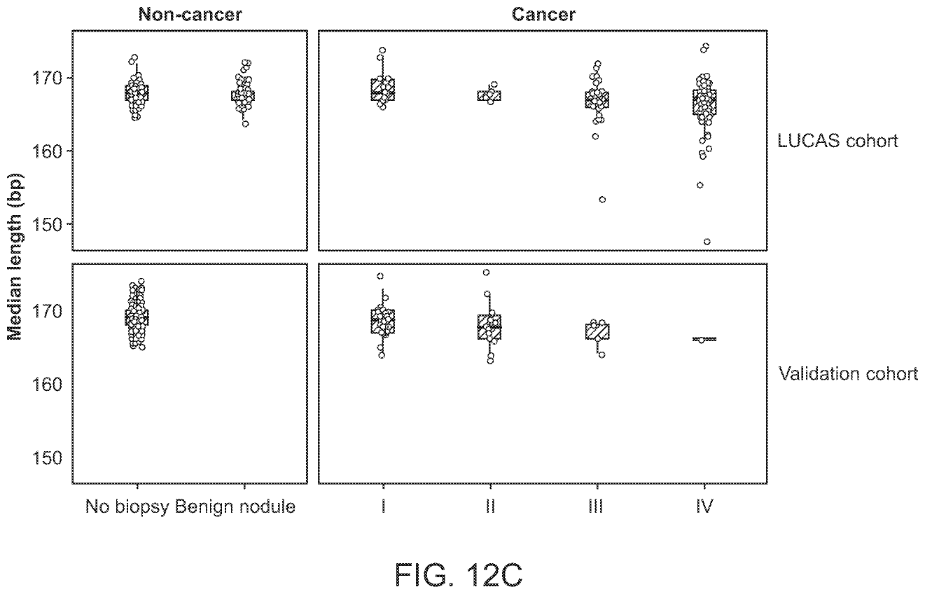

The relationship between DELFI scores and cancer stage and histology was examined next. While the DELFI score for non-cancer individuals was low (median DELFI scores of 0.16 or 0.21 for those without a biopsy or with benign lesions, respectively), patients with cancer had significantly higher median DELFI scores (DELFI scores for stage I=0.35, stage II=0.75, stage III=0.90, and stage IV=0.99)(p<0.01 for Stages I, II, III, or IV, Wilcoxon rank sum test) (FIG. 3A). A receiver operator characteristic (ROC) curve representing sensitivity and specificity of the DELFI approach to identify cancer patients in the LUCAS cohort revealed an area under the curve (AUC) of 0.90 (95% CI=0.86-0.94) (FIG. 3B). Stage I disease was more difficult to identify (AUC=0.76) but stage II, III and IV disease had similarly high performances (AUC_{II}=0.89, AUC_{III}=0.92, AUC_{IV}=0.92, respectively). When considering detection of individuals without a prior history of cancer, consistent with the inclusion criteria of other cancer screening studies^{4,5}, a higher performance overall was observed (AUC=0.93, 95% CI=0.90-0.97) as well as for individual stages (AUC_{I}=0.89, AUC_{II}=0.89, AUC_{III}=0.93, AUC_{IV}=0.95) (FIG. 3B). Similarly, analyses of the subset of individuals in this group that were considered at high risk for lung cancer (50-80 years old, smoking history >=20 pack-years) revealed an overall AUC of 0.94 (FIG. 3B). Analyses of different histologic subtypes of lung cancer showed that small cell (SCLC) and squamous cell (SCC) lung cancers were more easily detected than lung adenocarcinoma (FIG. 3B). To evaluate the robustness of a prior multi-cancer DELFI approach, the features and machine learning approach of Cristiano et al.²³ in the current study and identified similar performances (AUC=0.87, 95% CI=0.82-0.91, for all patients, and AUC=0.90, 95% CI=0.86-0.94 for patients without a prior history of cancer) (FIG. 12A). Other whole genome analyses, such as ichorCNA³¹ which only includes copy number changes, and analyses of overall median cfDNA fragment lengths provided substantially weaker performance with overall AUCs of 0.76, (95% CI=0.70-0.82) and 0.61 (95% CI=0.54-0.67), respectively (FIG. 12B). The lower performance of bulk fragment lengths may reflect the inability of this metric to capture fragmentation changes across the genome (Spearman correlation=0.29).

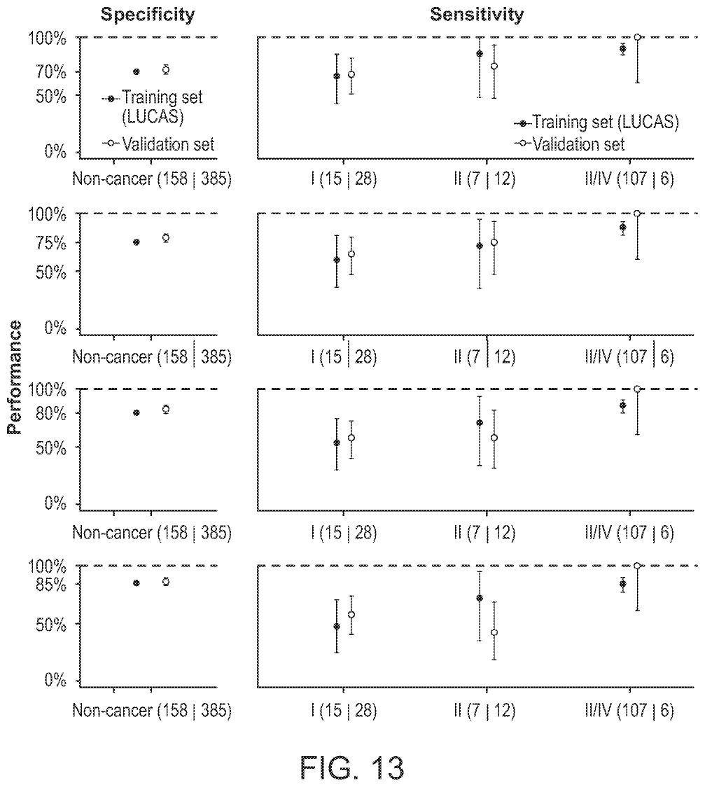

To externally validate the predictive performance of DELFI in an independent group of individuals with or without lung cancer, a single fragmentation-based model was first obtained using the non-cancer individuals and patients with baseline lung cancer in the LUCAS study and determined the DELFI score cutoff required to achieve specificities ranging from 70%-85%. Next, in an independent validation cohort comprised of individuals without cancer (n=385) or predominantly early stage cancer (n=46), the fixed model in LUCAS was used to compute DELFI scores in the validation set. Using the previously established cutoffs, the cancer status for individuals in the validation set was predicted according to whether their DELFI score was above or below the cutoff. The sensitivities and specificities of this model in the validation cohort were similar to those observed in the LUCAS cohort at different stages of the disease and among different histologic subtypes (FIG. 3C, FIG. 13). Overall, these analyses provide that the DELFI approach is generalizable across different lung cancer cohorts, including across different stages and histologic subtypes.

Combining DELFI Profiles with Multimodal Analyses

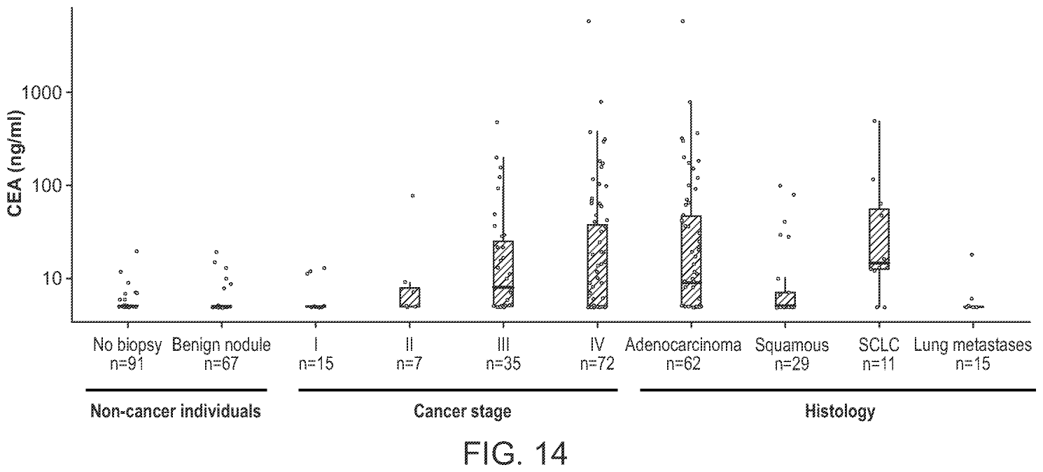

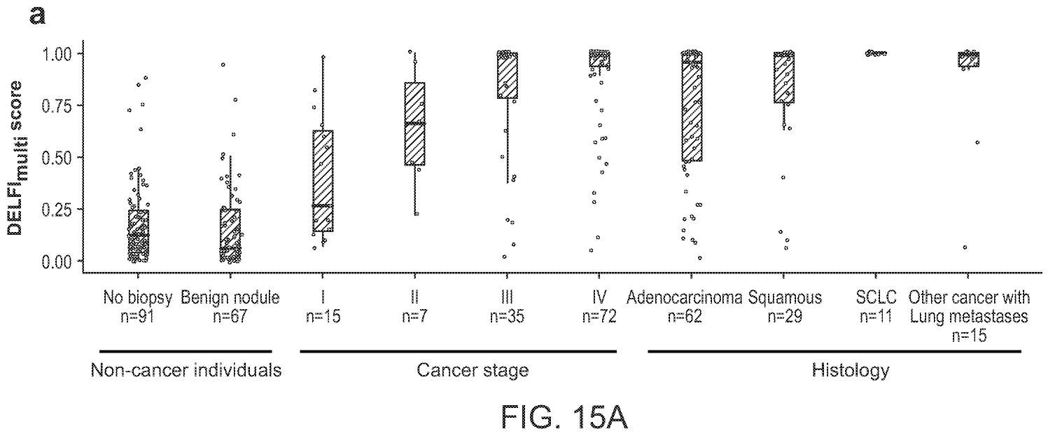

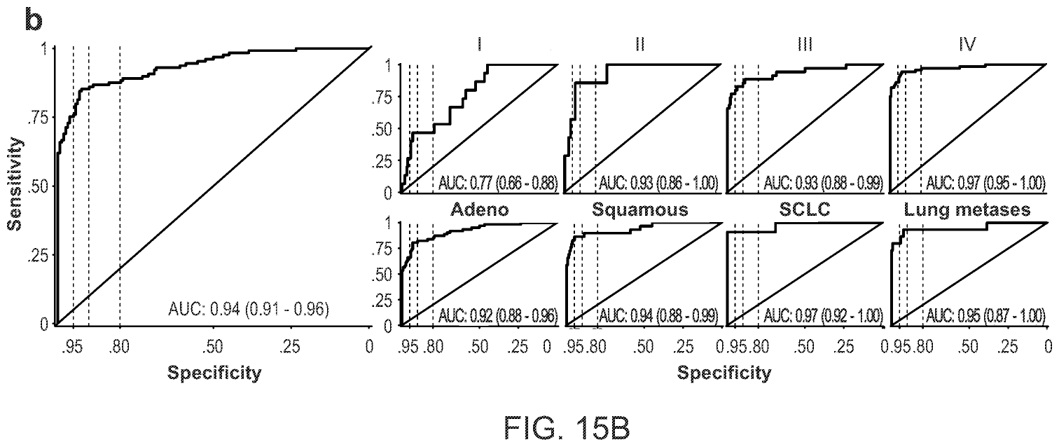

To evaluate multimodal approaches for cancer detection in combination with the multi-feature cfDNA analyses, the serum levels of carcinoembryonic antigen (CEA), a secreted protein that has been proposed as a lung biomarker^{12,15,32,33} was first assessed. Patients with lung cancer had higher CEA levels compared to patients without cancer, with more than 20% of stage I-III and the majority of stage IV cancer patients detected at levels >7.5 ng/ml, while only ˜4% of non-cancer patients fell above this threshold12,15,34 (p<0.001)(FIG. 14). CEA levels increased with stage, and patients with adenocarcinoma and SCLC subtypes showed higher levels compared to those with SCC or metastases to the lung (FIG. 14). As clinical characteristics have been proposed as risk factors for lung and other cancers³⁵, the genome-wide cfDNA fragmentation features were combined with CEA levels, age, smoking history, and presence of COPD in a multimodal model (DELFIₘᵤₗₜᵢ) (see Methods). Repeated cross-validation was used to predict whether these multimodal features represented characteristics of non-cancer individuals or cancer patients. Assessment of performance of the DELFIₘᵤₗₜᵢ revealed an overall AUC of 0.93, for individual stages (AUC_{I}=0.78, AUC_{II}=0.95, AUC_{III}=0.94, AUC_{IV}=0.95), and across histologic subtypes (AUC_{adeno}=0.91, AUC_{squamous}=0.95; AUC_{SCLC}=0.96) (FIGS. 15A, 15B). Although this approach could not be evaluated in the validation cohort, these analyses provide evidence that the combination of DELFI with a serum protein and clinical risk factors improves DELFI performance compared to those obtained through fragmentation profiles alone.

DELFI Analyses of Lung Cancer Progression and Outcome

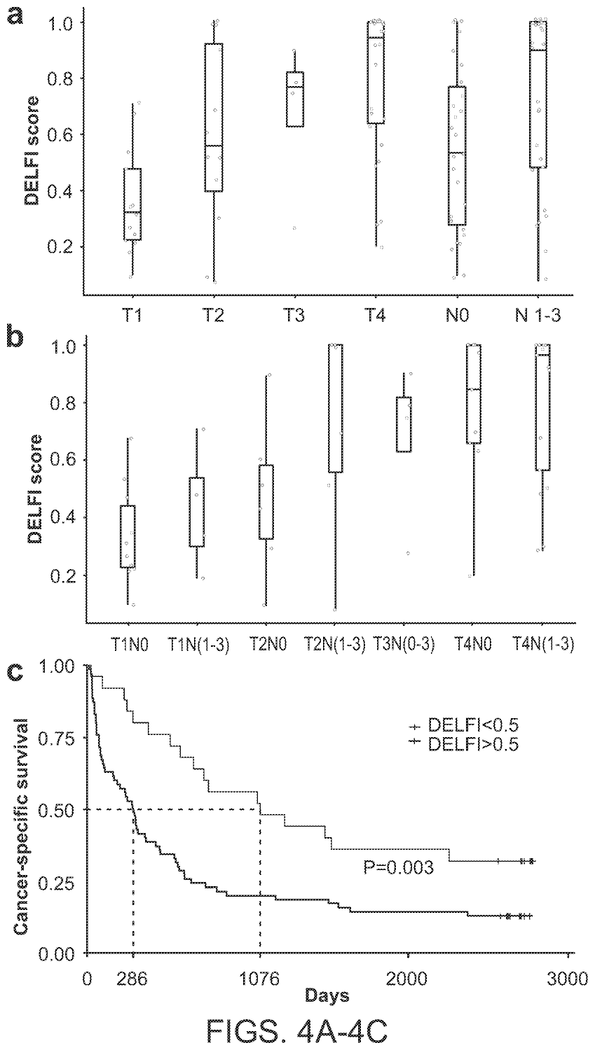

To examine the relationship between fragmentation profiles and lung tumor progression it was assessed whether the size of the lung cancer lesion or other clinical or radiological findings were related to aberrant fragmentation profiles. Although previous studies suggest small tumors (e.g. ˜1 cm³) may be missed by mutation based approaches given the limited number of ctDNA molecules at specific locations and limits of detection with these methods of ˜0.1%²⁰, genome-wide approaches may allow for more sensitive detection of such changes. As the DELFI approach interrogates ˜40 million fragments, it was expected that ˜40,000 fragments across the genome would be tumor derived in a patient with a small tumor having a 0.1% ctDNA contribution, thereby increasing the chances of detection. Interestingly in the LUCAS cohort, eight of the nine tumors less than two cm in size (T1a) had DELFI scores higher than the median non-cancer population (median DELFI score of 0.40 vs. 0.16) (FIG. 4A). Analyses of DELFI scores and T stage in patients with localized disease showed a stepwise increase of the DELFI scores from T1 to T4 stages (median DELFI scores for T1=0.32, T2=0.56, T3=0.77, T4=0.94; T1 vs T4, p<0.001, Wilcoxon rank sum test). Additionally, lung cancer patients without nodal involvement (NO) had a significantly lower DELFI score compared to the patients with lymph node metastases (FIG. 4A, p<0.001, Wilcoxon test). A stepwise increase in DELFI scores was also observed when assessing T and N stages in affected patients (FIG. 4B, p=0.005). These observations indicate a direct relationship between lung tumor size and aberrant fragmentation profiles in the circulation and provide evidence that even relatively small tumors may be detectable, including as previously observed in cases that were undetectable using deep-targeted sequencing (˜30,000×)²³.

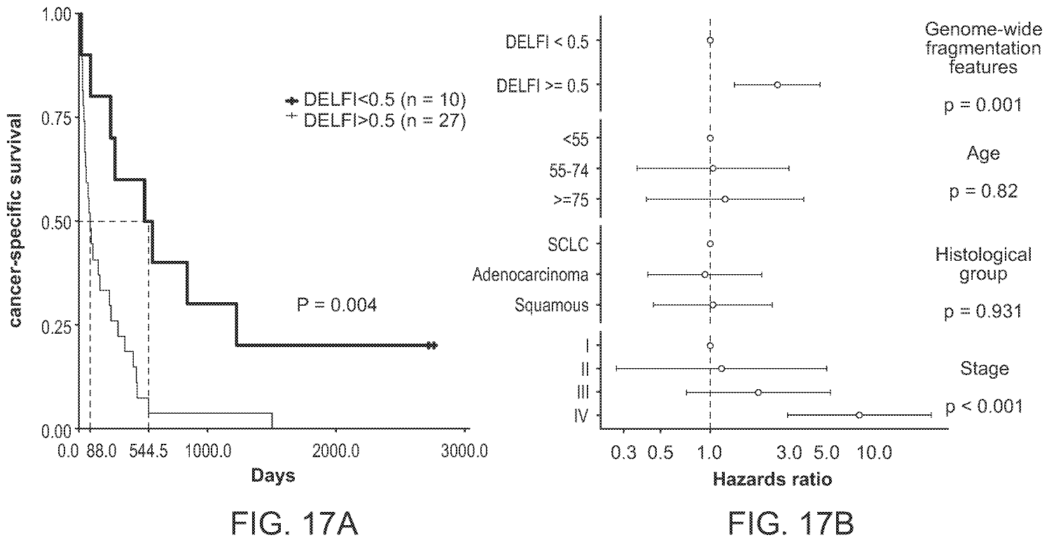

The long clinical follow-up of the LUCAS cohort (7-8 years) enabled an analysis of the association between DELFI scores and survival. These analyses revealed that a DELFI score greater than 0.5 was associated with a decreased overall survival compared to DELFI scores below 0.5 (P<0.001, FIG. 4C). In a multivariable analysis using a Cox proportional hazards model, the association of DELFI scores with survival was independent of cancer histology and stage with a hazard ratio (HR) of 2.53 (p=0.001, FIG. 17B). Similar results were obtained when analyzing a homogenous population of patients with stage IV adenocarcinoma (p=0.004, FIG. 17A), or when using other DELFI score thresholds ranging from 0.3 to 0.9. The DELFI score remained an independent prognostic factor even when considering differences in therapy among these individuals (P=0.04, HR=2.3) or when excluding patients that had a short survival. These results substantiate the relationship between fragmentation patterns and tumor burden or aggressiveness, and may provide clinical insights into long-term lung cancer outcomes.

Given the important differences in biologic characteristics and clinical management of SCLC and NSCLC, it was evaluated whether genome-wide fragmentation profiles could be used to noninvasively distinguish between these cancer types. Publicly available TCGA RNA-seq data from lung cancer subtypes was used to identify transcription factors with the highest differential expression between SCLC (n=79) and NSCLC (n=1046) or white blood cell (n=755) samples, and identified ASCL1 (Achaete-Scute Family basic helix-loop-helix Transcription Factor 1) as the gene most highly differentially expressed (>960 fold compared to NSCLC and WBC) (FIG. 5A). ASCL1 is a pioneer transcription factor in neuroendocrine cells, the progenitor cell type of SCLC, and has been identified to be overexpressed in the majority of SCLCs30. As expected, a subset of the genes with ASCL1 binding sites were differentially expressed between SCLC and NSCLC (FIG. 5B). Given the reported differences in cfDNA coverage at regions of transcription factor binding³⁶, it was evaluated whether fragment coverage and size across the observed 13,000 genome-wide binding sites of ASCL1 were altered in cfDNA of SCLC patients. A remarkable and consistent decrease in aggregate fragment coverage was observed at regions containing the ASCL1 binding sites (+/−200 bp) of patients with SCLC compared to non-cancer individuals or those with other cancer types (FIG. 5C). In contrast, at distances further from ASCL1 binding sites (>2000 bp), the fragment coverage between SCLC and other patients were similar. cfDNA fragment sizes in regions of ASCL1 binding were larger, presumably reflecting the decreased contribution of the tumor derived cfDNA which is typically smaller than non-cancer cfDNA23 (FIGS. 16A, 16C). Using fragment information in the ASCL1 binding regions a classifier was created that could be used to accurately detect 10 of 11 SCLCs (91% sensitivity, 95% CI=65%-99%) compared to 158 non-cancer individuals at >99% specificity (95% CI=98%-100%) (AUC=0.92) (FIG. 5D). Additionally, when considering DELFI positive cases (DELFI score >0.34 corresponding to an 80% specificity), SCLC patients were classified compared to other DELFI positive cases without SCLC with high accuracy (100% sensitivity, 95% CI-78%-100% at 95% specificity, 95% CI=90%-98%, AUC=0.98) (FIG. 5E). Despite the limited number of SCLC cases, these findings provide evidence that fragmentation profiles can reflect cell type-specific genome-wide transcription factor binding and provide a non-invasive approach for distinguishing lung cancers with different histologic subtypes.

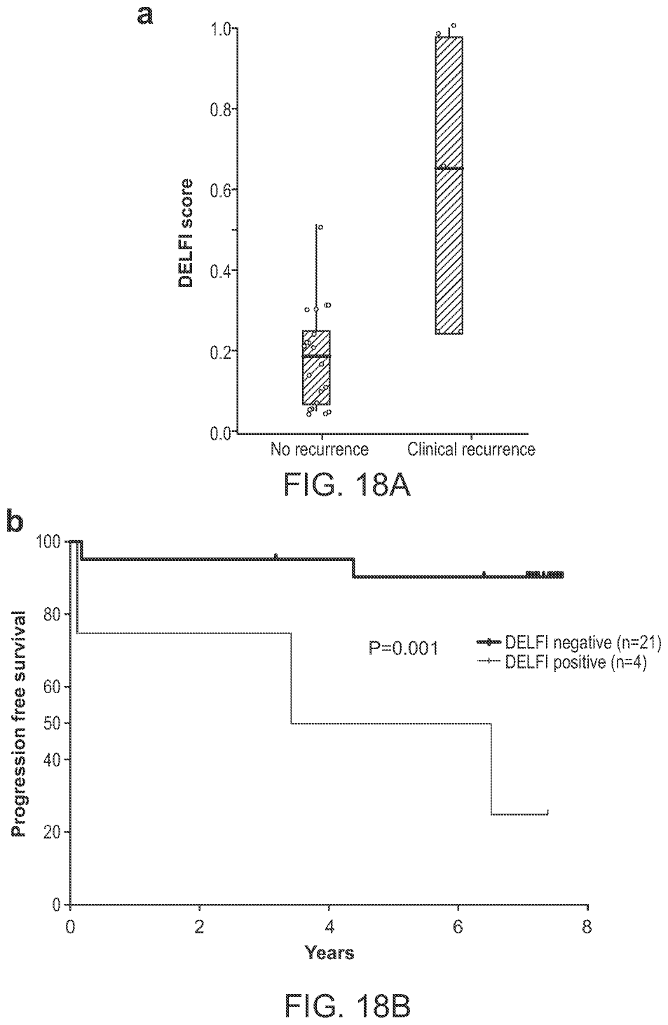

Analyses of patients with a previous history of cancer who were in clinical remission at the time of the DELFI baseline assessment identified 25 patients, five who recurred, and four who ultimately died from this disease. These included three patients with head and neck cancers, one with colon cancer, and one with malignant melanoma. Patients with subsequent recurrence had significantly higher DELFI scores than those individuals without recurrence (median DELFI scores 0.65 vs 0.19, p=0.005) (FIG. 18A). Additionally patients who scored positive on the DELFI test (DELFI score >0.34) had significantly shorter relapse-free survival (time from blood draw to relapse) compared to patients with negative DELFI test scores (p<0.01, FIG. 18B). These results support the notion that the DELFI approach can be used for detection of disease recurrence after treatment.

Additionally, the longitudinal clinical follow-up available in the LUCAS cohort enabled an analysis of fragmentation profiles in individuals who were deemed cancer-free at baseline but who developed a new cancer after baseline assessment. Of the 17 study subjects with a subsequent cancer diagnosis within two years (excluding localized skin tumors), four patients had DELFI scores greater than 0.5 at the time of enrollment, ranging from 0.5 to 1.0 within 33 to 481 days after enrollment. The malignancies identified comprised one case of NSCLC, as well as three non-pulmonary malignancies including chronic lymphocytic leukemia (CLL) and two B cell lymphomas. These data provide evidence that elevated DELFI scores may identify the emergence of cancers that were clinically undetected.

To evaluate the theoretical impact of a non-invasive molecular blood test on lung cancer detection, the performance of the DELFI score or the multimodal DELFImulti score was examined, followed by standard diagnostic CT imaging in the LUCAS cohort. This would allow examining the scenario where high-risk individuals would first have a blood draw and, depending on the results of the cfDNA analyses, individuals follow the pathway of either having an LDCT if the DELFI score is positive or not having an LDCT if the score is negative (FIG. 6A). Analysis of the performance of LDCT alone in the LUCAS cohort demonstrated high sensitivity (>95%) and a low specificity (58%). In a model where the DELFI score would have been used to prescreen patients, and only those that were positive were further evaluated by LDCT, the observed sensitivity of the combined DELFI/LDCT approach would be 90% (stage I=80%, stage II=86%, stage III=94, and stage IV=90%) and provide an increase in specificity to 80% (FIG. 6B). The DELFIₘᵤₗₜᵢ approach followed by LDCT would have improved the sensitivity to 94% overall (stage I=87%, stage II=100%, stage III=97%, and stage IV=96%) at the same specificity, and would have decreased the number of unnecessary procedures from 67 with LDCT alone to 32 (52% reduction) when using the combined approach (FIG. 6B).

To examine how the approach herein would perform for the overall detection of individuals with lung cancer at a population scale, the DELFI model was evaluated in a theoretical population of 100,000 high-risk individuals using Monte Carlo simulations. Using the estimated sensitivities and specificities of LDCT alone or with DELFI as a prescreen in this hypothetical population (FIG. 6B), the uncertainty of these parameters were modeled using probability distributions centered at empirical estimates obtained from the NLST and/or LUCAS cohorts (FIG. 6C). The likely prevalence of lung cancer in this population using the NLST study 5 estimate of 0.91% would be 910 individuals (95% CI, 428-1,584). Despite the recommendations for LDCT screening, adherence in the US is only 5.9%⁷, resulting in an average of 5979 individuals tested (95% CI, 3,176-9,658). As blood tests offer high accessibility and compliance, with adherence rates of 80-90% reported for blood-based biomarkers^{37,38}, it was assumed that an average of 60% (95% CI, 39%-76%) of the lung cancer screening population would be tested using the combined approach. Monte Carlo simulations from these probability distributions revealed that LDCT alone detected an average of 51 individuals (95% CI, 17-108) with lung cancer (FIG. 6D). Using DELFI as a prescreen for LDCT, on average 394 additional lung cancer cases were detected, or an ˜8-fold increase (95% CI, 4.4- to 19.6-fold increase) compared to LDCT alone (FIG. 6D). The combined approach would not only substantially improve detection of lung cancer, but would be expected to increase the accuracy of the test, reduce the number of unnecessary procedures, and increase positive predictive value (PPV) from 1.9% for LDCT_{LUCAS} and 2.6% for LDCT_{NLST} (95% CI, 0.8-3.8%) to 3.9% for DELFI and LDCT (95% CI, 1.8-7.9%, FIGS. 6E-6G). These analyses suggest a significant population-wide benefit for combining a high-sensitivity blood-based early detection test with a subsequent diagnostic LDCT for detection of lung cancer.

### Discussion

Overall, an improved DELFI approach is described for genome-wide fragmentation analyses for detection of lung cancer. It is proposed that facile and scalable analyses of the cfDNA fragmentome could be used to prescreen high-risk populations for lung cancer to increase accessibility of lung cancer detection and decrease unnecessary follow-up imaging procedures and invasive biopsies. Through the analysis of the LUCAS cohort, it was demonstrated that the DELFI approach can detect lung cancer across all stages and histologic subtypes compared to non-cancer individuals with or without benign lung nodules. The validation of the fixed DELFI model from the LUCAS cohort in an independent validation cohort supports the generalizability of the approach. Similar to observations with targeted sequencing approaches^{16,22,39-43}, the relationship between DELFI scores and tumor progression and long-term mortality provides evidence that the blood based fragmentation analyses may identify occult disease not observed by imaging, or more accurately identify the aggressiveness of the disease. The distinction between NSCLC and SCLC may allow for noninvasive characterization and treatment of lung cancer patients when tissues are not available. The identification of patients by DELFI that were only identified months later to have cancer through standard diagnostic methods shows the utility of the approach for cancer detection, detection of recurrent disease, and the potential for detection of cancers at earlier stages (“stage shifting”) through lung cancer screening. The possibility of combining a genome-wide multi-feature fragmentation profile analyses with a standard protein marker and clinical characteristics provides an avenue for high complexity multimodal analyses that can further increase the sensitivity of the approach.

Despite the publication of the NLST trial almost a decade ago⁵, the impact of LDCT in reducing lung cancer morbidity and mortality has been limited. Challenges for this approach have included insufficient imaging facilities and infrastructure that can screen large numbers of patients, the complexity of the medical workup that requires frequent visits and shared decision making, and repetitive radiation exposure from annual screening⁴⁴. Additionally, imaging studies detect radiographic abnormalities, not cancer, and result in biopsy-identified cancer diagnoses in only a small minority of positive scan findings, while the majority of false positive findings may drive invasive diagnostic procedures as well as ongoing patient anxiety during months or years of follow-up. Finally, while screening has been recognized as an important step for early detection of lung cancer in high-risk individuals, a significant percentage of lung cancer occurs in lower risk individuals⁴⁵ and current USPSTF recommendations do not recommend LDCT screening for these patients due to the imbalance of harms and benefits.

The analyzed cohorts represent real-world, prospective populations and the collection and processing of all samples were performed in a systematic fashion, ensuring homogeneity of pre-analytical characteristics and careful control of experimental and analytical variables. The potential improvement of the positive predictive value in the combined LDCT/DELFI approach suggests that many fewer unnecessary procedures would be performed in individuals with positive results. Additionally, the DELFI score appears to not be affected by non-cancer conditions, which have confounded other potential biomarkers for lung cancer detection. The observations that scalable and cost-effective non-invasive cfDNA fragmentation analyses can discriminate lung cancer patients from non-cancer individuals may ultimately provide an opportunity to evaluate not only high-risk individuals but the general population for lung cancer.

TABLE 1
Patient demographics and clinical information in LUCAS cohort

| | Non-cancer | Lung cancer | | | |
|---|---|---|---|---|---|
| | individuals | patients | | | |
| Patient Characteristic | n = 236 | n = 129 | P-value* | Lung lesion histology | n |
| Age | | | | Benign | 87 |
| Mean | 63 | 69 | <0.001 | Adenocarcinoma | 62 |
| Range | 19-96 | 33-94 | | Squamous cell carcinoma | 29 |
| Sex | | | | Small cell | 11 |
| Male | 125 | 61 | 0.3 | Adenosquamous | 3 |
| Female | 111 | 68 | | NSCLC, not otherwise specified | 3 |
| Smoking pack-years | | | | Mixed small cell and NSCLC | 1 |
| Mean | 26 | 42 | <0.001 | Mesothelioma | 1 |
| Range | 0-110 | 0-150 | | Neuroendocrine | 1 |
| Never smoker | 45 | 7 | | Metastasis from other organ | 15 |
| Current smoker | 71 | 51 | | Unknown | 3 |
| Quit >6 months | 96 | 55 | 0.004 | Stage | |
| Quit <6 months | 24 | 13 | | IA | 11 |
| Unknown | — | 3 | | IB | 4 |
| History of cancer | | | | IIA | 2 |
| None | 183 | 94 | | IIB | 5 |
| Prior cancer (<5 yrs) | 23 | 16 | | IIIA | 17 |
| Prior cancer (>5 yrs) | 27 | 13 | 0.31 | IIIB | 15 |
| Prior cancer (<5 yrs and >5 yrs) | 4 | 6 | | IIIC | 3 |
| Prior lung cancer | 2 | 5 | | IV | 72 |

*P-values were calculated to compare data from individuals with and without lung cancer for the following variables: mean ages and smoking pack years using Student's unpaired 2-tailed t-tests, sex distribution using a χ2 test, and smoking status and history of cancer using one-way ANOVAs.

TABLE 2
Patient demographics and clinical information for validation cohort

| | No lung | Lung | | | |
|---|---|---|---|---|---|
| Patient | cancer | cancer | | | |
| characteristics | n = 385 | n = 46 | P-value* | Lung lesion histology | n |
| Age | | | | Adenocarcinoma | 27 |
| Mean | 59 | 59 | 0.56 | Large cell carcinoma | 9 |
| Range | 50-75 | 38-76 | | Squamous cell carcinoma | 7 |
| Sex | | | | Adenosquamous | 1 |
| Male | 177 | 33 | 0.001 | Small cell | 1 |
| Female | 208 | 13 | | Mixed small cell and NSCLC | 1 |
| | | | | Stage | |
| | | | | I | 28 |
| | | | | II | 12 |
| | | | | III | 5 |
| | | | | IV | 1 |

*P-values were calculated to compare data from individuals with and without lung cancer for the following variables: mean ages using Student's unpaired 2-tailed t-tests and sex distribution using a χ2 test.

### REFERENCES

1. Ferlay J, Colombet M, Soerjomataram I, et al. Estimating the global cancer incidence and mortality in 2018: GLOBOCAN sources and methods. International journal of cancer 2019; 144:1941-53.
2. De Angelis R, Sant M, Coleman M P, et al. Cancer survival in Europe 1999-2007 by country and age: results of EUROCARE—5-a population-based study. The Lancet Oncology 2014; 15:23-34.
3. de Groot P M, Wu C C, Carter B W, Munden R F. The epidemiology of lung cancer. Translational lung cancer research 2018; 7:220-33.
4. de Koning H J, van der Aalst C M, de Jong P A, et al. Reduced Lung-Cancer Mortality with Volume CT Screening in a Randomized Trial. The New England journal of medicine 2020; 382:503-13.
5. National Lung Screening Trial Research T, Aberle D R, Adams A M, et al. Reduced lung-cancer mortality with low-dose computed tomographic screening. The New England journal of medicine 2011; 365:395-409.
6. Richards T B, Soman A, Thomas C C, et al. Screening for Lung Cancer—10 States, 2017. MMWR Morbidity and mortality weekly report 2020; 69:201-6.
7. Lung cancer Screening. 2020. at https://progressreport.cancer.gov/detection/lung_cancer.)
8. Pinsky P F. Principles of Cancer Screening. The Surgical clinics of North America 2015; 95:953-66.
9. Mazzone P J, Sears C R, Arenberg D A, et al. Evaluating Molecular Biomarkers for the Early Detection of Lung Cancer: When Is a Biomarker Ready for Clinical Use? An Official American Thoracic Society Policy Statement. American journal of respiratory and critical care medicine 2017; 196:e15-e29.
10. Chaturvedi A K, Caporaso N E, Katki H A, et al. C-reactive protein and risk of lung cancer. Journal of clinical oncology: official journal of the American Society of Clinical Oncology 2010; 28:2719-26.
11. Tang H, Bai Y, Shen W, et al. Clinical significance of combined detection of interleukin-6 and tumour markers in lung cancer. Autoimmunity 2018; 51:191-8.
12. Integrative Analysis of Lung Cancer E, Risk Consortium for Early Detection of Lung C, Guida F, et al. Assessment of Lung Cancer Risk on the Basis of a Biomarker Panel of Circulating Proteins. JAMA oncology 2018; 4:e182078.
13. Tang Z M, Ling Z G, Wang C M, Wu Y B, Kong J L. Serum tumor-associated autoantibodies as diagnostic biomarkers for lung cancer: A systematic review and meta-analysis. PloS one 2017; 12:e0182117.
14. Silvestri G A, Vachani A, Whitney D, et al. A Bronchial Genomic Classifier for the Diagnostic Evaluation of Lung Cancer. The New England journal of medicine 2015; 373:243-51.
15. Seijo L M, Peled N, Ajona D, et al. Biomarkers in Lung Cancer Screening: Achievements, Promises, and Challenges. Journal of thoracic oncology: official publication of the International Association for the Study of Lung Cancer 2019; 14:343-57.
16. Phallen J, Sausen M, Adleff V, et al. Direct detection of early-stage cancers using circulating tumor DNA. Science translational medicine 2017; 9.
17. Liu M C, Oxnard G R, Klein E A, Swanton C, Seiden M. Response to W. C. Taylor, and C. Fiala and E. P. Diamandis. Annals of oncology: official journal of the European Society for Medical Oncology 2020; 31:1268-70.
18. Lennon A M, Buchanan A H, Kinde I, et al. Feasibility of blood testing combined with PET-CT to screen for cancer and guide intervention. Science 2020; 369.
19. Chabon J J, Hamilton E G, Kurtz D M, et al. Integrating genomic features for non-invasive early lung cancer detection. Nature 2020; 580:245-51.
20. Abbosh C, Birkbak N J, Wilson G A, et al. Phylogenetic ctDNA analysis depicts early-stage lung cancer evolution. Nature 2017; 545:446-51.
21. Shen S Y, Singhania R, Fehringer G, et al. Sensitive tumour detection and classification using plasma cell-free DNA methylomes. Nature 2018; 563:579-83.
22. Leal A, van Grieken N C T, Palsgrove D N, et al. White blood cell and cell-free DNA analyses for detection of residual disease in gastric cancer. Nature communications 2020; 11:525.
23. Cristiano S, Leal A, Phallen J, et al. Genome-wide cell-free DNA fragmentation in patients with cancer. Nature 2019; 570:385-9.
24. MacMahon H, Naidich D P, Goo J M, et al. Guidelines for Management of Incidental Pulmonary Nodules Detected on CT Images: From the Fleischner Society 2017. Radiology 2017; 284:228-43.
25. Patel V K, Naik S K, Naidich D P, et al. A practical algorithmic approach to the diagnosis and management of solitary pulmonary nodules: part 1: radiologic characteristics and imaging modalities. Chest 2013; 143:825-39.
26. Patel V K, Naik S K, Naidich D P, et al. A practical algorithmic approach to the diagnosis and management of solitary pulmonary nodules: part 2: pretest probability and algorithm. Chest 2013; 143:840-6.
27. Benjamini Y, Speed T P. Summarizing and correcting the GC content bias in high-throughput sequencing. Nucleic acids research 2012; 40:e72.
28. Cancer Genome Atlas Research N. Comprehensive molecular profiling of lung adenocarcinoma. Nature 2014; 511:543-50.
29. Cancer Genome Atlas Research N. Comprehensive genomic characterization of squamous cell lung cancers. Nature 2012; 489:519-25.
30. George J, Lim J S, Jang S J, et al. Comprehensive genomic profiles of small cell lung cancer. Nature 2015; 524:47-53.
31. Adalsteinsson V A, Ha G, Freeman S S, et al. Scalable whole-exome sequencing of cell-free DNA reveals high concordance with metastatic tumors. Nature communications 2017; 8:1324.
32. Gropp C, Lehmann F G, Havemann K. [Carcinoembryonic antigen (CEA) in patients with lung cancer: correlation with tumour extent and response to treatment (author's transl)]. Deutsche medizinische Wochenschrift 1977; 102:1079-82.
33. Grunnet M, Sorensen JB. Carcinoembryonic antigen (CEA) as tumor marker in lung cancer. Lung cancer 2012; 76:138-43.
34. Hanash S M, Ostrin E J, Fahrmann J F. Blood based biomarkers beyond genomics for lung cancer screening. Translational lung cancer research 2018; 7:327-35.
35. Tammemagi M C, Katki H A, Hocking W G, et al. Selection criteria for lung-cancer screening. The New England journal of medicine 2013; 368:728-36.
36. Ulz P, Perakis S, Zhou Q, et al. Inference of transcription factor binding from cell-free DNA enables tumor subtype prediction and early detection. Nature communications 2019; 10:4666.
37. Bokhorst L P, Alberts A R, Rannikko A, et al. Compliance Rates with the Prostate Cancer Research International Active Surveillance (PRIAS) Protocol and Disease Reclassification in Noncompliers. European urology 2015; 68:814-21.
38. Duffy M J, van Rossum L G, van Turenhout S T, et al. Use of faecal markers in screening for colorectal neoplasia: a European group on tumor markers position paper. International journal of cancer 2011; 128:3-11.
39. Phallen J, Leal A, Woodward B D, et al. Early Noninvasive Detection of Response to Targeted Therapy in Non-Small Cell Lung Cancer. Cancer research 2019; 79:1204-13.
40. Anagnostou V, Forde P M, White J R, et al. Dynamics of Tumor and Immune Responses during Immune Checkpoint Blockade in Non-Small Cell Lung Cancer. Cancer research 2019; 79:1214-25.
41. Nabet B Y, Esfahani M S, Moding E J, et al. Noninvasive Early Identification of Therapeutic Benefit from Immune Checkpoint Inhibition. Cell 2020; 183:363-76 e13.
42. Bratman S V, Yang C S Y, Iafolla M A J, et al. Personalized circulating tumor DNA analysis as a predictive biomarker in solid tumor patients treated with pembrolizumab. Nat Cancer 2020; 1: 873-881.
43. Zhang Q, Luo J, Wu S, et al. Prognostic and Predictive Impact of Circulating Tumor DNA in Patients with Advanced Cancers Treated with Immune Checkpoint Blockade. Cancer discovery 2020; 10:1842-53.
44. Jemal A, Fedewa S A. Lung Cancer Screening With Low-Dose Computed Tomography in the United States-2010 to 2015. JAMA oncology 2017; 3:1278-81.
45. Kang H R, Cho J Y, Lee S H, et al. Role of Low-Dose Computerized Tomography in Lung Cancer Screening among Never-Smokers. Journal of thoracic oncology: official publication of the International Association for the Study of Lung Cancer 2019; 14:436-44.
46. Chen S, Zhou Y, Chen Y, Gu J. fastp: an ultra-fast all-in-one FASTQ preprocessor. Bioinformatics 2018; 34:i884-i90.
47. Langmead B, Salzberg S L. Fast gapped-read alignment with Bowtie 2. Nature methods 2012; 9:357-9.
48. Tarasov A, Vilella A J, Cuppen E, Nijman I J, Prins P. Sambamba: fast processing of NGS alignment formats. Bioinformatics 2015; 31:2032-4.
49. Quinlan A R, Hall I M. BEDTools: a flexible suite of utilities for comparing genomic features. Bioinformatics 2010; 26:841-2.
50. Leary R J, Sausen M, Kinde I, et al. Detection of chromosomal alterations in the circulation of cancer patients with whole-genome sequencing. Science translational medicine 2012; 4:162ra54.
51. P. KMaB. RTCGA: The Cancer Genome Atlas Data Integration. R package version 1.16.0.2019.
52. Davoli T, Uno H, Wooten E C, Elledge S J. Tumor aneuploidy correlates with markers of immune evasion and with reduced response to immunotherapy. Science 2017; 355.
53. Vansteenkiste J, De Ruysscher D, Eberhardt W E, et al. Early and locally advanced non-small-cell lung cancer (NSCLC): ESMO Clinical Practice Guidelines for diagnosis, treatment and follow-up. Annals of oncology: official journal of the European Society for Medical Oncology 2013; 24 Suppl 6:vi89-98.
54. Sorensen M, Pijls-Johannesma M, Felip E, Group EGW. Small-cell lung cancer: ESMO Clinical Practice Guidelines for diagnosis, treatment and follow-up. Annals of oncology: official journal of the European Society for Medical Oncology 2010; 21 Suppl 5:v120-5.
55. D'Addario G, Fruh M, Reck M, et al. Metastatic non-small-cell lung cancer: ESMO Clinical Practice Guidelines for diagnosis, treatment and follow-up. Annals of oncology: official journal of the European Society for Medical Oncology 2010; 21 Suppl 5:v116-9.
56. L C-T. Explore and download data from the recount3 project. 2021.
57. Jiang L, Huang J, Higgs B W, et al. Genomic Landscape Survey Identifies SRSF1 as a Key Oncodriver in Small Cell Lung Cancer. PLoS genetics 2016; 12:e1005895.
58. Lambert S A, Jolma A, Campitelli L F, et al. The Human Transcription Factors. Cell 2018; 172:650-65.
59. Hokari S, Tamura Y, Kaneda A, et al. Comparative analysis of TTF-1 binding DNA regions in small-cell lung cancer and non-small-cell lung cancer. Molecular oncology 2020; 14:277-93.
60. Robin X, Turck N, Hainard A, et al. pROC: an open-source package for R and S+ to analyze and compare ROC curves. BMC bioinformatics 2011; 12:77.
61. Borromeo M D, Savage T K, Kollipara R K, et al. ASCL1 and NEUROD1 Reveal Heterogeneity in Pulmonary Neuroendocrine Tumors and Regulate Distinct Genetic Programs. Cell reports 2016; 16:1259-72.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

1. A method of cancer diagnosis in a subject, comprising:
extracting cell free (cfDNA) from the subject's biological sample;
generating genomic libraries from the extracted cfDNA and whole genome sequencing of cfDNA fragments;
mapping of the cfDNA fragments to a genomic origin and evaluating fragment length and obtaining genome-wide fragmentation profiles for each sample;
identifying protein biomarkers of the subject;
comparing the subject's cfDNA fragmentation profile and protein biomarkers with normal reference non-cancer subjects;
and,
diagnosing cancer in a subject.

2. The method of claim 1, wherein the cancer is lung cancer.

3. The method of claim 1, further comprising subjecting the subject to a low dose helical computed tomography (LDCT).

4. The method of claim 1, further comprising comparing clinical data between the subject diagnosed as having lung cancer and normal non-cancer subjects.

5. The method of claim 1, wherein the cfDNA fragment mean length and profiles are similar among non-cancer individuals.

6. The method of claim 1, wherein the cfDNA fragment profiles of cancer subjects vary.

7. The method of claim 1, wherein serum levels of or one or more tumor antigens, cytokines or proteins are measured.

8. The method of claim 7, wherein one or more tumor antigens are measured and comprise: carcinoembryonic antigen (CEA), CA19-9, CA 125, tissue polypeptideantigen (TSA), CYFRA-21-1, neuron-specific enolase, progastrin-releasing peptide (ProGRP), plasma kalikrein B1 (KLKB1), serum amyloid A, haptoglobin-alpha-2, ADAM-17, osteoprotegerin, pentraxin 3, follistatin, tumor necrosis factor receptor superfamily member 1A or combinations thereof.

9. The method of claim 7, wherein the one or more proteins are measured and comprise C-reactive protein (CRP), Chitinase-3-like protein 1 (YKL-40/CHI3L1) or fragments thereof.

10. The method of claim 1, wherein DNA evaluation of fragments for early interception (DELFI) is conducted to produce a DELFI score.

11. The method of claim 10, wherein the DELFI scores for non-cancer individuals are less than about 0.3.

12. The method of claim 10, wherein the DELFI scores for stage I cancer are between about 0.3 to less than 0.5

13. The method of claim 10, wherein the DELFI scores for stage II cancer are between about 0.5 to less than 0.8.

14. The method of claim 10, wherein the DELFI scores for stage III cancer are between about 0.8 to less than 0.95.

15. The method of claim 10, wherein the DELFI scores for stage IV cancer are about 0.95 or greater.

16. The method of claim 10, wherein the DELFI score for stage I cancer is about 0.35.

17. The method of claim 10, wherein the DELFI score for stage II cancer is about 0.75.

18. The method of claim 10, wherein the DELFI score for stage III cancer is about 0.9.

19. The method of claim 10, wherein the DELFI score for stage IV cancer is about 0.99.

20. The method of claim 1, wherein the subject is administered cancer therapies.

21. A method of diagnostically distinguishing between subjects with small cell lung cancer (SCLC) from those with non-small cell lung cancer (NSCLC) or without cancer, the method comprising:
comparing differential expression of transcription factors in biological samples of SCLC, NSCLC or white blood cells;
selecting at least one or more transcription factors having a higher differential expression as compared to the expression of transcription factors identified in the biological samples;
extracting cell free (cfDNA) from the subject's biological sample;
obtaining genome-wide fragmentation profiles of the cfDNA obtained from the subject to identify the at least one or more transcription factor binding sites;
evaluating cfDNA coverage of the at least one or more transcription factor binding sites to determine fragment coverage and size as compared to non-cancer subjects or NSCLC subjects; thereby,
diagnostically distinguishing between subjects with small cell lung cancer (SCLC) from those with non-small cell lung cancer (NSCLC) or without cancer.

22. The method of claim 21, wherein the at least one transcription factor is Achaete-Scute Family basic helix-loop-helix Transcription Factor 1 (ASCL1).

23. The method of claim 22, wherein the cfDNA fragment sizes in nucleic acid sequences comprising ASCL1 binding sites are larger in SCLC patients as compared to patients with NSCLC or non-cancer subjects.

24. The method of claim 22, wherein aggregate fragment coverage in nucleic acid sequences comprising ASCL1 binding sites is decreased in SCLC patients as compared to patients with NSCLC or non-cancer subjects.

25. A method of diagnostically distinguishing between subjects with small cell lung cancer (SCLC) from those with non-small cell lung cancer (NSCLC) or without cancer, the method comprising:
extracting cell free (cfDNA) from the subject's biological sample;
evaluating cfDNA coverage of Achaete-Scute Family basic helix-loop-helix Transcription Factor 1 (ASCL1) binding sites to determine fragment coverage and size as compared to non-cancer subjects or NSCLC subjects; thereby,
diagnostically distinguishing between subjects with small cell lung cancer (SCLC) from those with non-small cell lung cancer (NSCLC) or without cancer.

26-29. (canceled)

30. A method of determining recurrence of cancer in a subject comprising the method of claim 1.

31. A method of correcting GC content of a genome-wide fragmentation analyses, comprising:
sequencing of whole genome libraries of cancer subjects and cancer-free subjects from samples not subjected to polymerase chain reaction (PCR) and samples subjected to a variable number of PCR cycles,
filtering of adapter sequences,
aligning sequence reads against a human reference genome and removing of duplicate reads,
converting each aligned pair to a genomic interval, wherein the genomic interval represents sequenced DNA fragments, and
selecting reads having a mapq score of at least 30 or greater.

32-40. (canceled)
